(19) United States
(12) Patent Application Publication
(10) Pub. No.: US 2024/0327440 A1
(43) Pub. Date: Oct. 3, 2024

(54) METHOD FOR PREPARING FLUOROPHORE- AND CHROMOPHORE-CONTAINING CONJUGATES FOR VISUAL DETECTION OF ANALYTE MOLECULES

(71) Applicant: SONY GROUP CORPORATION, Tokyo (JP)

(72) Inventors: Melissa F. Jackson, Bothell, WA (US); Tracy Matray, Snohomish, WA (US)

(21) Appl. No.: 18/570,283

(22) PCT Filed: Jun. 16, 2022
(86) PCT No.: PCT/IB2022/055558

§ 371 Date: Dec. 14, 2023

Related U.S. Application Data
(60) Provisional application No. 63/211,435, filed on Jun. 16, 2021.

(51) Int. Cl. C07F 9/6561; C09B 69/10; C09K 11/06; G01N 33/58

(52) U.S. Cl. CPC C07F 9/6561; C09B 69/10; C09K 11/06; G01N 33/582; C09K 2211/1044

(57) ABSTRACT

Methods of preparing compounds useful as water soluble dyes are disclosed. Specifically, the compounds have the following Structure (I): (I) or a stereoisomer, tautomer or salt thereof, wherein L¹, L², L³, R¹, R², and M are as defined herein.

### BACKGROUND

### Field

The present disclosure is generally directed to preparation of fluorophore and chromophore containing compounds (e.g., polymer compounds comprising fluorescent dye moieties) and their use in various analytical methods.

### Description of the Related Art

Fluorescent and/or colored dyes are known to be particularly suitable for applications in which a highly sensitive detection reagent is desirable. Dyes that are able to preferentially label a specific analyte molecule, ingredient, or component in a sample enable the researcher to determine the presence, quantity and/or location of that specific analyte molecule, ingredient, or component. In addition, specific systems can be monitored with respect to their spatial and temporal distribution in diverse environments.

Fluorescence and colorimetric methods are extremely widespread in chemistry and biology. These methods give useful information on the presence, structure, distance, orientation, complexation and/or location for biomolecules. In addition, time-resolved methods are increasingly used in measurements of dynamics and kinetics. As a result, many strategies for bio-conjugation of fluorescence or color labels to biomolecules, such as nucleic acids and protein, have been developed. Since analysis of biomolecules typically occurs in an aqueous environment, the focus has been on development and use of dyes compatible with aqueous systems that can elucidate desired spatial information and biomolecule interactions.

Accordingly, there is a need in the art for preparing such water soluble dyes for bio-conjugation, especially dyes having an increased molar brightness. Ideally, compounds comprising fluorophores and chromophores are prepared to yield relatively pure products that cleanly conjugate to a desired analyte molecule, ingredient, or component. The present disclosure fulfills this need and provides further related advantages.

### BRIEF SUMMARY

In brief, embodiments of the present disclosure are generally directed to preparation of compounds useful as water soluble, fluorescent and/or colored dyes and/or probes that enable visual detection of analyte molecules, such as biomolecules. In particular, one embodiment provides a method of preparing a compound, the method comprising contacting a compound of Structure (I) with 1,4,-dithiothreitol, the compound of Structure (I) having the following structure:

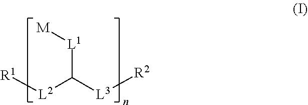

wherein R¹, R², L¹, L², L³, and M are as defined herein. These and other aspects of the disclosure will be apparent upon reference to the following detailed description.

### DETAILED DESCRIPTION

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “about” has the meaning reasonably ascribed to it by a person of ordinary skill in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or 1% of the stated value.

“Amino” refers to the —NH₂ group.

“Carboxy” refers to the —CO₂H group.

“Sulfhydryl,” “thiol,” or “thio” refers to the —SH group.

“Thioxo” refers to the ═S group.

“Hydroxyl” or “hydroxyl” refers to the —OH group.

“Alkyl” refers to a straight or branched hydrocarbon chain group consisting solely of carbon and hydrogen atoms, containing no unsaturation, having from one to twelve carbon atoms (C₁-C₁₂ alkyl), one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, alkyl groups are optionally substituted.

“Hydroxylalkyl” refers to any alkyl group as defined above wherein at least one hydrogen is replaced with a hydroxyl group. Unless stated otherwise specifically in the specification, a hydroxylalkyl group is optionally substituted.

“Aminoalkyl” refers to any alkyl group as defined above wherein at least one hydrogen is replaced with an amino group. Unless stated otherwise specifically in the specification, an aminoalkyl group is optionally substituted.

“Sulfhydrylalkyl” refers to any alkyl group as defined above wherein at least one hydrogen is replaced with a sulfhydryl group. Unless stated otherwise specifically in the specification, a sulfhydrylalkyl group is optionally substituted.

“Alkylether” refers to any alkyl group as defined above, wherein at least one carbon-carbon bond is replaced with a carbon-oxygen bond. The carbon-oxygen bond may be on the terminal end (as in an alkoxy group) or the carbon oxygen bond may be internal (i.e., C—O—C). Alkylethers include at least one carbon oxygen bond, but may include more than one. For example, polyethylene glycol (PEG) is included within the meaning of alkylether. Unless stated otherwise specifically in the specification, an alkylether group is optionally substituted. For example, in some embodiments an alkylether is substituted with an alcohol or —OP(═Rₐ)(R_{b})R_{c}, wherein each of Rₐ, R_{b} and R_{c} is as defined for compounds of Structure (I).

“Hydroxyalkylether” refers to an alkylether group as defined above, wherein at least one hydrogen is replaced with a hydroxyl group. Unless stated otherwise specifically in the specification, a hydroxylalkylether group is optionally substituted.

“Sulfhydrylalkylether” refers to any alkylether group as defined above wherein at least one hydrogen is replaced with a sulfhydryl group. Unless stated otherwise specifically in the specification, a sulfhydrylalkylether group is optionally substituted.

“Alkoxy” refers to a group of the formula —ORₐ where Rₐ is an alkyl group as defined above containing one to twelve carbon atoms. Unless stated otherwise specifically in the specification, an alkoxy group is optionally substituted.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, alkylene is optionally substituted.

“Heteroalkylene” refers to an alkylene group, as defined above, comprising at least one heteroatom (e.g., Si, N, O, P, or S) within the alkylene chain or at a terminus of the alkylene chain. In some embodiments, the heteroatom is within the alkylene chain (i.e., the heteroalkylene comprises at least one carbon-[heteroatom]-carbon bond, where x is 1, 2, or 3). In other embodiments, the heteroatom is at a terminus of the alkylene and thus serves to join the alkylene to the remainder of the molecule (e.g., Mₐ-H-A-M_{b}, where Mₐ and M_{b} are each a separate portion of the molecule, H is a heteroatom, and A is an alkylene). Unless stated otherwise specifically in the specification, a heteroalkylene group is optionally substituted. Exemplary heteroalkylene groups include ethylene oxide (e.g., polyethylene oxide) and the “C linker,” “HEG linker,” and “PEG 1K linker” linking groups illustrated below:

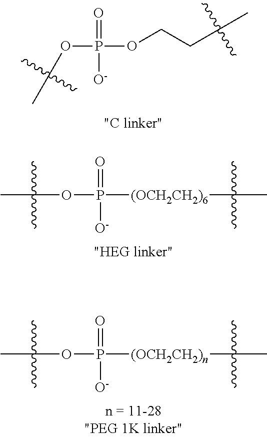

Multimers of the above C linker, HEG linker, and/or PEG 1K linker are included in various embodiments of heteroalkylene linkers. In some embodiments of the PEG 1K linker, n ranges from 19-25, for example n is 19, 20, 21, 22, 23, 24, or 25. Multimers may comprise, for example, the following structure:

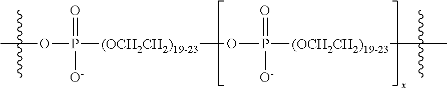

wherein x is 0 or an integer greater than 0, for example, x ranges from 0-100 (e.g., 1, 2, 3, 4, 5, 6, 7, 8, 9 or 10).

“Heteroatomic” in reference to a “heteroatomic linker” refers to a linker group consisting of one or more heteroatom. Exemplary heteroatomic linkers include single atoms selected from the group consisting of O, N, P, and S, and multiple heteroatoms for example a linker having the formula —P(O⁻)(═O)O— or —OP(O⁻)(═O)O— and multimers and combinations thereof.

“Phosphate” refers to the —OP(═O)(Rₐ)R_{b} group, wherein Rₐ is OH, O⁻ or OR_{c}; and R_{b} is OH, O⁻, OR_{c}, a thiophosphate group or a further phosphate group, wherein R_{c} is a counter ion (e.g., Na+ and the like).

“Phosphoalkyl” refers to the —OP(═O)(Rₐ)R_{b} group, wherein Rₐ is OH, O⁻ or OR_{c}; and R_{b} is —Oalkyl (or alkoxy), wherein R_{c} is a counter ion (e.g., Na⁺ and the like). Unless stated otherwise specifically in the specification, a phosphoalkyl group is optionally substituted. For example, in certain embodiments, the —Oalkyl moiety in a phosphoalkyl group is optionally substituted with one or more of hydroxyl, amino, sulfhydryl, phosphate, thiophosphate, phosphoalkyl, thiophosphoalkyl, phosphoalkylether or thiophosphoalkylether.

“Phosphoalkylether” refers to the —OP(═O)(Rₐ)R_{b} group, wherein Rₐ is OH, O⁻ or OR_{c}; and R_{b} is —Oalkylether, wherein R_{c} is a counter ion (e.g., Na⁺ and the like). Unless stated otherwise specifically in the specification, a phosphoalkylether group is optionally substituted. For example, in certain embodiments, the —Oalkylether moiety in a phosphoalkylether group is optionally substituted with one or more of hydroxyl, amino, sulfhydryl, phosphate, thiophosphate, phosphoalkyl, thiophosphoalkyl, phosphoalkylether, or thiophosphoalkylether.

“Thiophosphate” refers to the —OP(═Rₐ)(R_{b})R_{c} group, wherein Rₐ is O or S, R_{b} is OH, O⁻, S⁻, OR_{d} or SR_{d}; and R_{c} is OH, SH, O⁻, S⁻, OR_{d}, SR_{d}, a phosphate group or a further thiophosphate group, wherein R_{d} is a counter ion (e.g., Na⁺ and the like) and provided that: i) Rₐ is S; ii) R_{b} is S⁻ or SR_{d}; iii) R_{c} is SH, S— or SR_{d}; or iv) a combination of i), ii) and/or iii).

“Thiophosphoalkyl” refers to the —OP(═Rₐ)(R_{b})R_{c} group, wherein Rₐ is O or S, R_{b} is OH, O⁻, S⁻, OR_{d} or SR_{d}; and R_{c} is —Oalkyl, wherein R_{d} is a counter ion (e.g., Na+ and the like) and provided that: i) Rₐ is S; ii) R_{b} is S⁻ or SR_{d}; or iii) Rₐ is S and R_{b} is S⁻ or SR_{d}. Unless stated otherwise specifically in the specification, a thiophosphoalkyl group is optionally substituted. For example, in certain embodiments, the —Oalkyl moiety in a thiophosphoalkyl group is optionally substituted with one or more of hydroxyl, amino, sulfhydryl, phosphate, thiophosphate, phosphoalkyl, thiophosphoalkyl, phosphoalkylether or thiophosphoalkylether.

“Thiophosphoalkylether” refers to the —OP(═Rₐ)(R_{b})R_{c} group, wherein Rₐ is O or S, R_{b} is OH, O⁻, S⁻, OR_{d} or SR_{d}; and R_{c} is —Oalkylether, wherein R_{d} is a counter ion (e.g., Na⁺ and the like) and provided that: i) Rₐ is S; ii) R_{b} is S⁻ or SR_{d}; or iii) Rₐ is S and R_{b} is S⁻ or SR_{d}. Unless stated otherwise specifically in the specification, a thiophosphoalkylether group is optionally substituted. For example, in certain embodiments, the —Oalkylether moiety in a thiophosphoalkyl group is optionally substituted with one or more of hydroxyl, amino, sulfhydryl, phosphate, thiophosphate, phosphoalkyl, thiophosphoalkyl, phosphoalkylether or thiophosphoalkylether.

“Alkylphospho” refers to the —RP(═O)(Rₐ)R_{b} group, wherein R is an alkylene group, Rₐ is OH, O⁻ or OR_{c}; and R_{b} is —Oalkyl, wherein R_{c} is a counter ion (e.g., Na⁺ and the like). Unless stated otherwise specifically in the specification, an alkylphospho group may be optionally substituted. For example, in certain embodiments, the —Oalkyl moiety (R_{b}) in a alkylphospho group is optionally substituted with one or more of hydroxyl, amino, sulfhydryl, phosphate, thiophosphate, phosphoalkyl, thiophosphoalkyl, phosphoalkylether or thiophosphoalkylether. “—Oalkylphospho” refers to an alkylphospho group connected to the remainder of the molecule via an oxygen atom. Unless stated otherwise specifically in the specification, an Oalkylphospho group may be optionally substituted.

“Alkyletherphospho” refers to the —RP(═O)(Rₐ)R_{b} group, wherein R is an alkylene group, Rₐ is OH, O⁻ or OR_{c}; and R_{b} is —Oalkylether, wherein R_{c} is a counter ion (e.g., Na⁺ and the like). Unless stated otherwise specifically in the specification, an alkylphospho group may be optionally substituted. For example, in certain embodiments, the —Oalkylether moiety (R_{b}) in a alkyletherphospho group is optionally substituted with one or more of hydroxyl, amino, sulfhydryl, phosphate, thiophosphate, phosphoalkyl, thiophosphoalkyl, phosphoalkylether or thiophosphoalkylether. “—Oalkyletherphospho” refers to an alkyletherphospho group connected to the remainder of the molecule via an oxygen atom. Unless stated otherwise specifically in the specification, an Oalkyletherphospho group may be optionally substituted.

“Alkylthiophospho” refers to the —P(═Rₐ)(R_{b})R_{c}, group, wherein Rₐ is O or S, R_{b} is OH, O⁻, S⁻, OR_{d} or SR_{d}; and R_{c} is —Oalkyl, wherein R_{d} is a counter ion (e.g., Na⁺ and the like) and provided that: Rₐ is S or R_{b} is S⁻ or SR_{d}; or provided that Rₐ is S and R_{b} is S⁻ or SR_{d}. Unless stated otherwise specifically in the specification, an alkylthiophospho group may be optionally substituted. For example, in certain embodiments, the —Oalkyl moiety in an alkylthiophospho group is optionally substituted with one or more of hydroxyl, amino, sulfhydryl, phosphate, thiophosphate, phosphoalkyl, thiophosphoalkyl, phosphoalkylether or thiophosphoalkylether. “—Oalkylthiophospho” is an alkylthiophospho group connected to the remainder of the molecule via an oxygen atom. Unless stated otherwise specifically in the specification, an Oalkylthiophospho group may be optionally substituted.

“Alkyletherthiophospho” refers to the —P(═Rₐ)(R_{b})R_{c} group, wherein Rₐ is O or S, R_{b} is OH, O⁻, S⁻, OR_{d} or SR_{d}; and R_{c} is —Oalkylether, wherein R_{d} is a counter ion (e.g., Na⁺ and the like) and provided that: Rₐ is S or R_{b} is S⁻ or SR_{d}; or provided that Rₐ is S and R_{b} is S⁻ or SR_{d}. Unless stated otherwise specifically in the specification, an alkyletherthiophospho group may be optionally substituted. For example, in certain embodiments, the —Oalkylether moiety in an alkyletherthiophospho group is optionally substituted with one or more of hydroxyl, amino, sulfhydryl, phosphate, thiophosphate, phosphoalkyl, thiophosphoalkyl, phosphoalkylether or thiophosphoalkylether. “—Oalkyletherthiophospho” is an alkylthiophospho group connected to the remainder of the molecule via an oxygen atom. Unless stated otherwise specifically in the specification, an Oalkyletherthiophospho group may be optionally substituted.

“Conjugation” refers to the overlap of one p-orbital with another p-orbital across an intervening sigma bond. Conjugation may occur in cyclic or acyclic compounds. A “degree of conjugation” refers to the overlap of at least one p-orbital with another p-orbital across an intervening sigma bond. For example, 1, 3-butadine has one degree of conjugation, while benzene and other aromatic compounds typically have multiple degrees of conjugation. Fluorescent and colored compounds typically comprise at least one degree of conjugation.

“Bio-conjugation” or “bio-conjugate” and related variations refer to a chemical reaction strategy for forming a stable covalent bond between two molecules. The term “bio-conjugation” is generally used when one of the molecules is a biomolecule (e.g., an antibody), but can be used to describe forming a covalent bond with a non-biomolecule (e.g., a polymeric resin). The product or compound resulting from such a reaction strategy is a “conjugate,” “bio-conjugate” or a grammatical equivalent.

A “linker” refers to a contiguous chain of at least one atom, such as carbon, oxygen, nitrogen, sulfur, phosphorous and combinations thereof, which connects a portion of a molecule to another portion of the same molecule or to a different molecule, moiety or solid support (e.g., microparticle). Linkers may connect the molecule via a covalent bond or other means, such as ionic or hydrogen bond interactions.

The terms “visible” and “visually detectable” are used herein to refer to substances that are observable by visual inspection, without prior illumination, or chemical or enzymatic activation. Such visually detectable substances absorb and emit light in a region of the spectrum ranging from about 300 to about 900 nm. Preferably, such substances are intensely colored, preferably having a molar extinction coefficient of at least about 40,000, more preferably at least about 50,000, still more preferably at least about 60,000, yet still more preferably at least about 70,000, and most preferably at least about 80,000 M⁻¹ cm⁻¹. The compounds of the disclosure may be detected by observation with the naked eye, or with the aid of an optically based detection device, including, without limitation, absorption spectrophotometers, transmission light microscopes, digital cameras and scanners. Visually detectable substances are not limited to those which emit and/or absorb light in the visible spectrum. Substances which emit and/or absorb light in the ultraviolet (UV) region (about 10 nm to about 400 nm), infrared (IR) region (about 700 nm to about 1 mm), and substances emitting and/or absorbing in other regions of the electromagnetic spectrum are also included with the scope of “visually detectable” substances.

“Fluorescent” refers to a molecule which is capable of absorbing light of a particular frequency and emitting light of a different frequency. Fluorescence is well-known to those of ordinary skill in the art.

“Colored” refers to a molecule which absorbs light within the colored spectrum (i.e., red, yellow, blue and the like).

“FRET” or “Forster resonance energy transfer” refers to a physical interaction whereby energy from the excitation of one moiety (e.g., a first chromophore or “donor”) is transferred to an adjacent moiety (e.g., a second chromophore or “acceptor”). “FRET” is sometimes also used interchangeably with “fluorescence resonance energy transfer” (i.e., when each chromophore is a fluorescent moiety). Generally, FRET requires that (1) the excitation or absorption spectrum of the acceptor chromophore overlaps with the emission spectrum of the donor chromophore; (2) the transition dipole moments of the acceptor and donor chromophores are substantially parallel (i.e., at about 0° or 180°); and (3) the acceptor and donor chromophores share a spatial proximity (i.e., close to each other). The transfer of energy from the donor to the acceptor occurs through non-radiative dipole-dipole coupling and the distance between the donor chromophore and acceptor chromophore is generally much less than the wavelength(s) of light.

“Donor” or “donor chromophore” refers to a chromophore (e.g., a fluorophore) that is or can be induced into an excited electronic state and may transfer its excitation or absorbance energy to a nearby acceptor chromophore in a non-radiative fashion through long-range dipole-dipole interactions. Without wishing to be bound by theory, it is thought that the energy transfer occurs because the oscillating dipoles of the respective chromophores have similar resonance frequencies. A donor and acceptor that have these similar resonance frequencies are referred to as a “donor-acceptor pair(s),” which is used interchangeably with “FRET moieties,” “FRET pairs,” “FRET dyes,” or similar. In some embodiments, at least one M is donor chromophore.

“Acceptor” or “acceptor chromophore” refers to a chromophore (e.g., a fluorophore) to which excitation or absorbance energy from a donor chromophore is transferred via a non-radiative transfer through long-range dipole-dipole interaction. In some embodiments, at least one M is an acceptor chromophore. In some embodiments, each M is either a donor chromophore or an acceptor chromophore.

“Stoke's shift” refers to a difference between positions (e.g., wavelengths) of the band maxima of excitation or absorbance and emission spectra of an electronic transition (e.g., from excited state to non-excited state, or vice versa). In some embodiments, the compounds of Structure (I) have a Stoke's shift greater than 25 nm, greater than 30 nm, greater than 35 nm, greater than 40 nm, greater than 45 nm, greater than 50 nm, greater than 55 nm, greater than 60 nm, greater than 65 nm, greater than 70 nm, greater than 75 nm, greater than 80 nm, greater than 85 nm, greater than 90 nm, greater than 95 nm, greater than 100 nm, greater than 110 nm, greater than 120 nm, greater than 130 nm, greater than 140 nm, greater than 150 nm, greater than 160 nm, greater than 170 nm, greater than 180 nm, greater than 190 nm, or greater than 200 nm.

“J-value” is calculated as an integral value of spectral overlap between the emission spectrum of a donor chromophore and the excitation or absorbance spectrum of an acceptor chromophore. The emission spectrum of the donor chromophore is that which is generated when the donor chromophore is excited with a preferred excitation or absorbance wavelength. Preferred excitation or absorbance wavelengths for donor chromophores are at or near their respective excitation or absorbance maximum well known to a person of ordinary skill in the art (e.g., Pacific Blue has an excitation or absorbance maximum at about 401 nm, FITC has an excitation or absorbance maximum at about 495 nm).

“Cyanine dye” of “tetramethylindo(di)-carbocyanines” are organic dye moieties with the general formula:

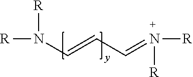

wherein:
R is at each occurrence, independently an optionally substituted alkyl, wherein 1 or more R groups may join to form a heterocyclic or heteroaryl ring or ring system; and
y is an integer greater than 0. For example, in some embodiments, y is 1, 2, 3, 4, 5, 6, 7, 8, 9, or 10. In some more specific embodiments, y is 1, 2, 3, 4, 5, or 6. In more specific embodiments, y is 1, 2, 3, or 4. In some embodiments, y is 1 or 2.

Cyanine dyes include streptocyanines (or open chain cyanines), hemicyanines, closed chain cyanines, neutrocyanines, merocyanines (e.g., spiropyrans and quinophthalones), apocyanines. As noted above, both nitrogens of the cyanine dye may be each independently part of a heteroaryl group such as pyrrolyl, imidazolyl, thiazolyl, pyridinyl, quinolinyl, indolyl, benzothiazolyl, etc. In some embodiments, derivatives of cyanine dyes may include the addition of functional groups selected from the group consisting of alkyl groups (e.g., methyl, ethyl, butyl, etc.), carboxy, acetylmethoxy, sulfo, and combinations thereof. Exemplary cyanine dyes include the following as well as reported excitation and emission wavelengths:

| | Excitation | Emission |
|---|---|---|
| Dye | Wavelength (nm) | Wavelength (nm) |
| Cy2 | 489 | 506 |
| Cy3 | (512); 550 | 570; (620) |
| Cy3B | 558 | 572; (620) |
| Cy3.5 | 581 | 594; (640) |
| Cy5 | (625); 650 | 670 |
| Cy5.5 | 675 | 694 |
| Cy7 | 743 | 767 |

Embodiments of this disclosure are also meant to encompass all compounds of Structure (I) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²p, ³⁵S, ¹⁸F, ³⁶Cl, ¹²³I, and ¹²⁵I, respectively.

Isotopically-labeled compounds of Structure (I) or (II) can generally be prepared by conventional techniques known to those skilled in the art or by processes analogous to those described below and in the following Examples using an appropriate isotopically-labeled reagent in place of the non-labeled reagent previously employed.

“Stable compound” and “stable structure” are meant to indicate a compound that is sufficiently robust to survive isolation to a useful degree of purity from a reaction mixture, and formulation into an efficacious therapeutic agent.

“Optional” or “optionally” means that the subsequently described event or circumstances may or may not occur; such a description includes instances where the event or circumstance occurs and instances where it does not. For example, “optionally substituted alkyl” means that the alkyl group may or may not be substituted and that the description includes both substituted alkyl groups and alkyl groups having no substitution.

“Salt” includes both acid and base addition salts.

“Acid addition salt” refers to those salts which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Base addition salt” refers to those salts which are prepared from addition of an inorganic base or an organic base to the free acid. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Crystallizations may produce a solvate of the compounds described herein. Embodiments of the present disclosure include all solvates of the described compounds. As used herein, the term “solvate” refers to an aggregate that comprises one or more molecules of a compound of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. The compounds of the disclosure may be true solvates, while in other cases the compounds of the disclosure may merely retain adventitious water or another solvent or be a mixture of water plus some adventitious solvent.

Embodiments of the compounds of the disclosure (e.g., compounds of structure I or II), or their salts, tautomers or solvates may contain one or more stereocenters and may thus give rise to enantiomers, diastereomers, and other stereoisomeric forms that may be defined, in terms of absolute stereochemistry, as (R)- or (S)- or, as (D)- or (L)- for amino acids. Embodiments of the present disclosure are meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

A “tautomer” refers to a proton shift from one atom of a molecule to another atom of the same molecule. The present disclosure includes tautomers of any said compounds. Various tautomeric forms of the compounds are easily derivable by those of ordinary skill in the art.

The chemical naming protocol and structure diagrams used herein are a modified form of the I.U.P.A.C. nomenclature system, using the ACD/Name Version 9.07 software program and/or ChemDraw Ultra Version 11.0 software naming program (CambridgeSoft). Common names familiar to one of ordinary skill in the art are also used.

### Methods of Preparation

Methods of the present disclosure are useful as a means for preparing biological samples for detection of analyte molecules (e.g., biomolecules). Methods of the present disclosure provide compounds that are fully and reliably prepared for bio-conjugation reactions. Methods of the present disclosure are useful for preparing dye molecules as well as bio-conjugate molecules comprising the same (e.g., compounds bound to analyte molecules).

Reducing agents are used for reduction of disulfide bonds to form sulfydryl functional groups. Tris(2-carboxyethyl)phosphine (TCEP) is a trialkylphosphine that is soluble in water up to about 1.08 M (at pH 2.5). It has minimal solubility in organic solvents and works over a wide range of pHs (about pH 2-9). TCEP does not contain any thiol functional groups, so it is not necessary to remove residual reagent or byproducts when it is used in low concentrations.

1,4-dithiothreitol (also known as Cleland's reagent or DTT) is an unstable reducing agent (e.g., it can be oxidized by atmospheric oxygen) that is somewhat soluble in water (about 50 mg/mL) and soluble in ethanol, acetone, chloroform, and ether. DTT works at pH>7 and contains thiols, so it does have to be removed from reaction mixtures after use.

Accordingly, one embodiment provides a method of preparing a compound, the method comprising contacting a compound of Structure (I) with 1,4,-dithiothreitol, the compound of Structure (I) having the following structure:

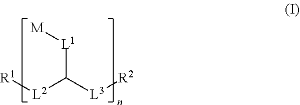

wherein:
M is, at each occurrence, independently a moiety comprising two or more double bonds and at least one degree of conjugation, provided that at least one occurrence of M is a cyanine or derivative thereof;
L¹, L², and L³ are, at each occurrence, independently optional linkers;
R¹ and R² are independently H, OH, SH, NH₂, alkyl, alkoxy, alkylether, hydroxylalkyl, aminoalkyl, hydroxylalkylether, sulfhydrylalkyl, —S—S-alkyl, —S—S-hydroxylalkyl, alkyl-S—S-alkyl, alkyl-S—S-hydroxylalkyl, phosphoalkyl-S—S-alkyl, phosphoalkyl-S—S-hydroxylalkyl, sulfyhdrylalkylether, phosphate, thiophosphate, alkylphospho, alkylthiophospho, —Oalkylphospho, —Oalkylthiophospho, alkyletherphospho, alkyletherthiophospho, —Oalkyletherphospho, —Oalkyletherthiophospho, phosphoalkyl, phosphoalkylether, thiophosphoalkyl, thiophosphoalkylether, —Ophosphoalkyl, —Ophosphoalkylether, —Othiophosphoalkyl, —Othiophosphoalkylether, or have the following structure:

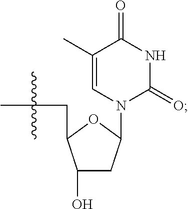 [SMILES: *CC1OC(n2cc(C)c(=O)[nH]c2=O)CC1O]

and
n is an integer greater than 0,
   provided that,
at least one of R¹-L²- or R²-L³- comprises a —S—S—* moiety, wherein * represents a terminal end of the compound of Structure (I).

In some embodiments, the contacting thereby converts the —S—S—* moiety to a —S—H moiety.

In certain embodiments, the contacting is performed in a solution having a pH between 6.5 and 8. In more specific embodiments, the contacting is performed in a solution having a pH between 7-7.5. In some embodiments, the contacting is performed in a solution having a pH greater than about 5.0, 5.5, 6.0, 6.5, 7.0, or 7.35. In some embodiments, the contacting is performed in a solution having a pH less than about 9.0, 8.5, 8.0, 7.9, 7.8, 7.75, or 7.6.

In some specific embodiments, the solution comprises ethylenediaminetetraacetic acid (EDTA). In still more embodiments, the solution comprises a concentration of ethylenediaminetetraacetic acid (EDTA) from about 1 to 6 mM. In certain specific embodiments, the solution comprises a concentration of ethylenediaminetetraacetic acid (EDTA) from about 1.5 to 5.5 mM. In some specific embodiments, the solution comprises a concentration of ethylenediaminetetraacetic acid (EDTA) from about 1.9 to 4.2 mM. In certain embodiments, the solution comprises a concentration of ethylenediaminetetraacetic acid (EDTA) from about 2 to 4 mM. In certain embodiments, the solution comprises a concentration of ethylenediaminetetraacetic acid (EDTA) greater than about 1.0, 1.5, 1.75, 1.8, 1.9, or 2.0 mM. In certain embodiments, the solution comprises a concentration of ethylenediaminetetraacetic acid (EDTA) less than about 6.0, 5.5, 5.0, 4.75, 4.5, or 4.0 mM.

In some embodiments, the solution comprises magnesium chloride (MgCl₂). In certain embodiments, the solution comprises magnesium chloride (MgCl₂) at a concentration between about 1 to 50 mM. In some specific embodiments, the solution comprises magnesium chloride (MgCl₂) at a concentration between about 2 to 40 mM. In certain specific embodiments, the solution comprises magnesium chloride (MgCl₂) at a concentration between about 3 to 35 mM. In some more specific embodiments, the solution comprises magnesium chloride (MgCl₂) at a concentration between about 4 to 30 mM. In certain more specific embodiments, the solution comprises magnesium chloride (MgCl₂) at a concentration between about 5 to 25 mM.

In some embodiments, the solution comprises an organic solvent. In more specific embodiments, the organic solvent is methanol. In other embodiments, the organic solvent is acetonitrile. In some embodiments, the organic solvent is a combination of methanol and acetonitrile. In some embodiments, the solution further comprises dimethylsulfoxide (DMSO).

In certain embodiments, the solution has an organic solvent concentration greater than about 5% by volume. In certain specific embodiments, the solution has an organic solvent concentration greater than about 7.5% by volume. In more specific embodiments, the solution has an organic solvent concentration greater than about 10% by volume. In some embodiments, the solution has an organic solvent concentration greater than about 15% by volume. In more specific embodiments, the solution has an organic solvent concentration greater than about 12%, 17%, 20%, 25%, 30%, 35%, 40%, 45%, 50%, 55%, 60%, 70%, 80%, 90% by volume.

In certain embodiments, the method further comprises contacting the —S—H moiety with a cross-linking reagent, thereby forming a covalent bond between the compound and the cross-linking reagent. In more specific embodiments, the cross-linking moiety comprises at least one maleimide moiety. In certain more specific embodiments, the cross-linking reagent has the following structure:

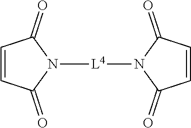

wherein:
L⁴ is a linker.

In certain specific embodiments, L⁴ comprises atoms selected from C and O. In some embodiments, L⁴ has one of the following structures:

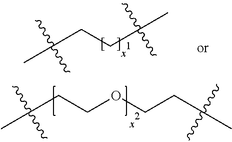

wherein:
x¹ and x² are an integer greater than 0.

In some embodiments, x¹ is 1. In more specific embodiments, x¹ is 3. In some embodiments, x¹ is 5. In some embodiments, x¹ is 1, 2, 3, 4, 5, 6, 7, or 8.

In certain embodiments, x² is 2. In more specific embodiments, x² is 3. In some embodiments, x² is 1, 2, 3, 4, 5, or 6.

In certain embodiments, the cross-linking reagent has one of the following structures:

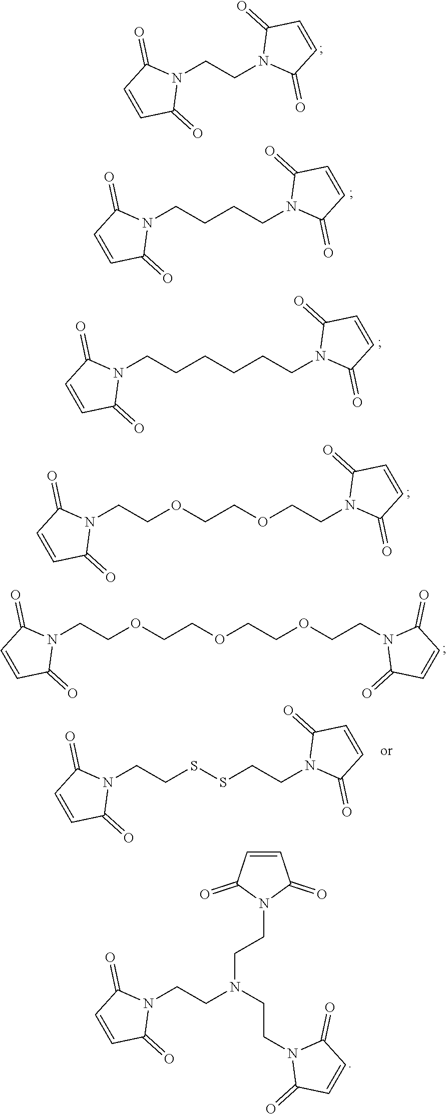

In certain embodiments, each occurrence of L¹ comprises atoms selected from C, O, S, N and P. In some embodiments, each occurrence of L² comprises atoms selected from C, O, S, N and P. In certain embodiments, each occurrence of L³ comprises atoms selected from C, O, S, N and P. In some embodiments, at least one occurrence of L¹ comprises atoms selected from C, O, and N.

In more specific embodiments, at least one occurrence of L¹ is an alkylene or heteroalkylene linker. In some embodiments, at least one occurrence of L¹ is an alkylene linker. In some embodiments, at least one occurrence of L¹ is a heteroalkylene linker. In more specific embodiments, at least one occurrence of L² has the following structure:

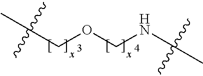

wherein:
x³ and x⁴ are each independently a integer greater than 0.

In some embodiments, x³ is 1, 2, 3, or 4. In some embodiments, x³ is 1, 2, 3, 4, 5, 6, 7, 8, 9, or 10. In certain embodiments, x⁴ is 2, 3, 4, or 5. In certain embodiments, x⁴ is 1, 2, 3, 4, 5, 6, 7, 8, 9, or 10. In more specific embodiments, x³ is 1 or 2 and x⁴ is 2, 3, or 4.

In certain embodiments, at least one occurrence of L¹ has one of the following structures:

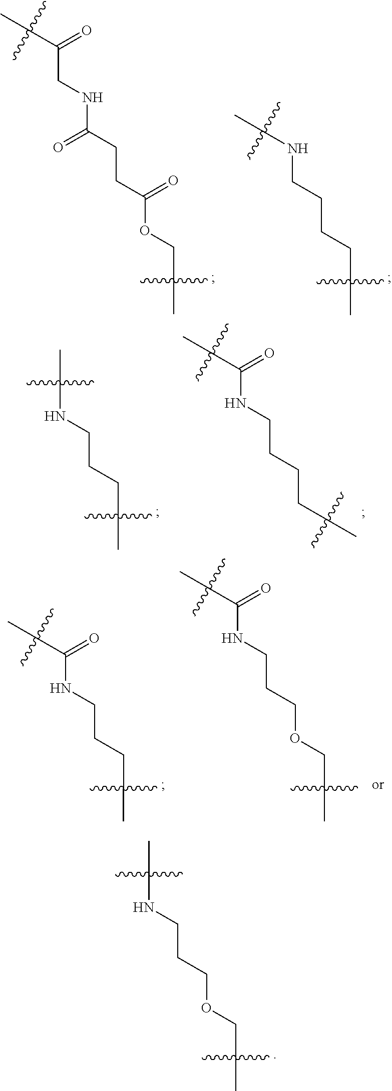

In some embodiments, each occurrence of L¹ has one of the following structures:

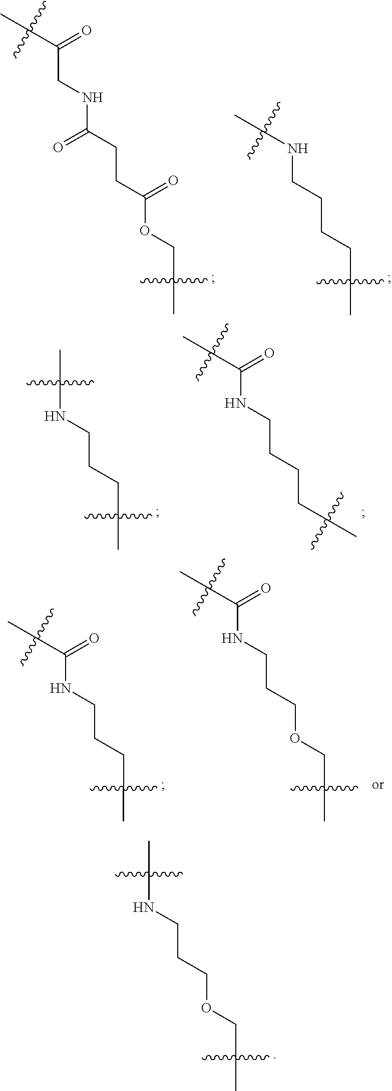

In some embodiments, n is an integer from 1-20. In more specific embodiments, n is an integer from 2-10. In some embodiments, n is 2, 3, 4, 5, 6, 7, 8, 9, or 10. In some embodiments, n is 3, 4, 5, 6, 7, 8, 9, or 10. In some embodiments, n is 4, 5, 6, 7, 8, 9, or 10. In certain embodiments, n is 5, 6, 7, 8, 9, 10, 11, or 12. In some embodiments, n is 6, 7, 8, 9, 10, 11, or 12.

In certain embodiments, at least one occurrence of L² comprises atoms selected from C, O, S, and P. In some embodiments, at least one occurrence of L² is alkylene or heteroalkylene.

In more specific embodiments, at least one occurrence of L² comprises the following structure:

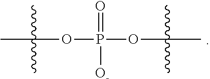

In some embodiments, at least one occurrence of L² comprises the following structure:

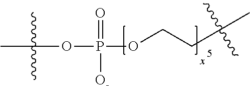

wherein:
x⁵ is an integer greater than 0.

In certain embodiments, x⁵ is 2, 3, or 6. In some embodiments, x⁵ is 1, 2, 3, 4, 5, 6, 7, 8, 9, 10, 11, or 12. In some embodiments, x⁵ ranges from 1-100. In some embodiments, x⁵ ranges from 3-30. In some embodiments, x⁵ ranges from 10-30. In some specific embodiments, x⁵ is 3 or 6.

In some embodiments, at least one occurrence of L² comprises the following structure:

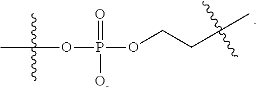

In some embodiments, at least one occurrence of L³ comprises atoms selected from C, O, S, and P. In more specific embodiments, at least one occurrence of L³ is alkylene or heteroalkylene.

In certain embodiments, at least one occurrence of L³ comprises the following structure:

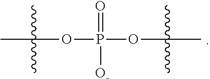

In some embodiments, at least one occurrence of L³ comprises the following structure:

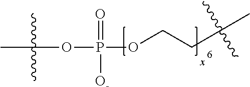

wherein:
x⁶ is an integer greater than 0.

In some embodiments, x⁶ is 2, 3, or 6. In some embodiments, x⁶ is 1, 2, 3, 4, 5, 6, 7, 8, 9, 10, 11, or 12. In some embodiments, x⁶ ranges from 1-100. In some embodiments, x⁶ ranges from 3-30. In some embodiments, x⁶ ranges from 10-30. In some specific embodiments, x⁶ is 3 or 6.

In certain embodiments, at least one occurrence of L³ comprises the following structure:

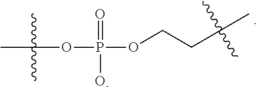

In certain embodiments, at least one occurrence of L³ comprises the following structure:

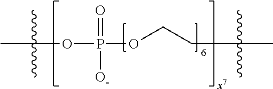

wherein:
x⁷ is an integer greater than 0. In some embodiments, x⁷ is 1, 2, 3, 4, 5, or 6. In more specific embodiments, x⁷ is 3.

In certain embodiments, at least one occurrence of L³ comprises the following structure:

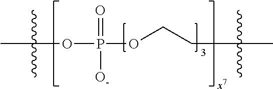

wherein:
x⁷ is an integer greater than 0. In some embodiments, x⁷ is 1, 2, 3, 4, 5, or 6. In more specific embodiments, x⁷ is 3.

In certain embodiments, at least one occurrence of L³ comprises the following structure:

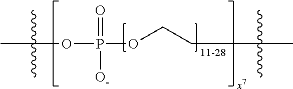

wherein:
x⁷ is an integer greater than 0. In some embodiments, x⁷ is 1, 2, 3, 4, 5, or 6. In more specific embodiments, x⁷ is 3. In some embodiments, x⁷ is 1.

In certain embodiments, R¹ is H, OH, SH, NH₂, alkyl, alkylether, hydroxylalkyl, aminoalkyl, hydroxylalkylether, sulfhydrylalkyl, —S—S-alkyl, —S—S-hydroxylalkyl, alkyl-S—S-alkyl, alkyl-S—S-hydroxylalkyl, phosphoalkyl-S—S-alkyl, phosphoalkyl-S—S-hydroxylalkyl, sulfyhdrylalkylether, phosphate, thiophosphate, or alkylphospho.

In some embodiments, R² is H, OH, SH, NH₂, alkyl, alkylether, hydroxylalkyl, aminoalkyl, hydroxylalkylether, sulfhydrylalkyl, —S—S-alkyl, —S—S-hydroxylalkyl, alkyl-S—S-alkyl, alkyl-S—S-hydroxylalkyl, phosphoalkyl-S—S-alkyl, phosphoalkyl-S—S-hydroxylalkyl, sulfyhdrylalkylether, phosphate, thiophosphate, alkylphospho, or has the following structure:

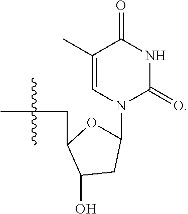 [SMILES: *CC1OC(n2cc(C)c(=O)[nH]c2=O)CC1O]

In some specific embodiments, R¹ comprises the following structure:

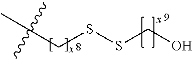

wherein:
x⁸ and x⁹ are each independently an integer greater than 0.

In some specific embodiments, x⁸ is 1, 2, 3, 4, 5, 6, 7, 8, 9, 10, 11, or 12. In more specific embodiments, x⁹ is 1, 2, 3, 4, 5, 6, 7, 8, 9, 10, 11, or 12. In some embodiments, x⁸ is 4, 5, 6, 7, or 8 and x⁹ is 4, 5, 6, 7, or 8.

In some more specific embodiments, at least one occurrence of M is a fluorophore or chromophore. In some embodiments, two or more occurrences of M form a FRET donor-acceptor pair. In some embodiments, at least one occurrence of M is a fluorescent or colored moiety. In some embodiments, at least one occurrence of M is a fluorescent or colored dye moiety. In some more embodiments, at least two occurrences of M are fluorescent or colored moieties. In some more embodiments, at least two occurrences of M are fluorescent or colored dye moieties.

In some embodiments, at least one occurrence of M is a moiety comprising three or more aryl or heteroaryl rings, or combinations thereof. In certain embodiments, at least one occurrence of M is a moiety comprising four or more aryl or heteroaryl rings, or combinations thereof. In some embodiments, the aryl or heteroaryl rings form a fused ring system.

In some embodiments, at least two occurrence of M are moieties comprising three or more aryl or heteroaryl rings, or combinations thereof. In certain embodiments, at least two occurrence of M are moieties comprising four or more aryl or heteroaryl rings, or combinations thereof. In some embodiments, the aryl or heteroaryl rings form a fused ring system; for example, in some embodiments, M is a pyrene dye, which is considered a moiety comprising 4 aryl rings as a fused ring system.

In some embodiments, M is, at each occurrence, independently selected from the group consisting of coumarin dye, resorufin dye, dipyrrometheneboron difluoride dye, ruthenium bipyridyl dye, thiazole orange dye, polymethine, N-aryl-1,8-naphthalimide dye, boron-dipyrromethene, rhodamine, a cyanine dye, pyrene, perylene, perylene monoimide, 6-FAM, 5-FAM, 6-FITC, 5-FITC, and derivatives thereof.

In certain embodiments, at least one occurrence of M has one of the following structures:

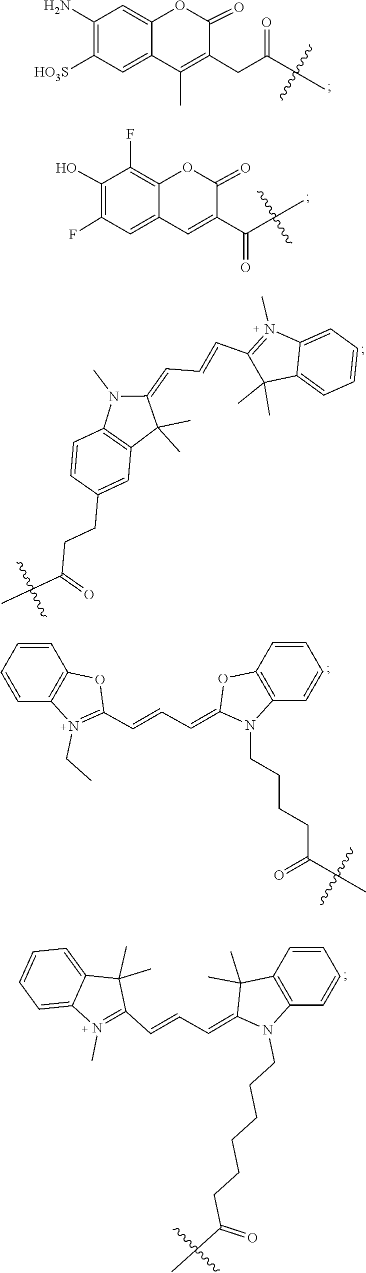

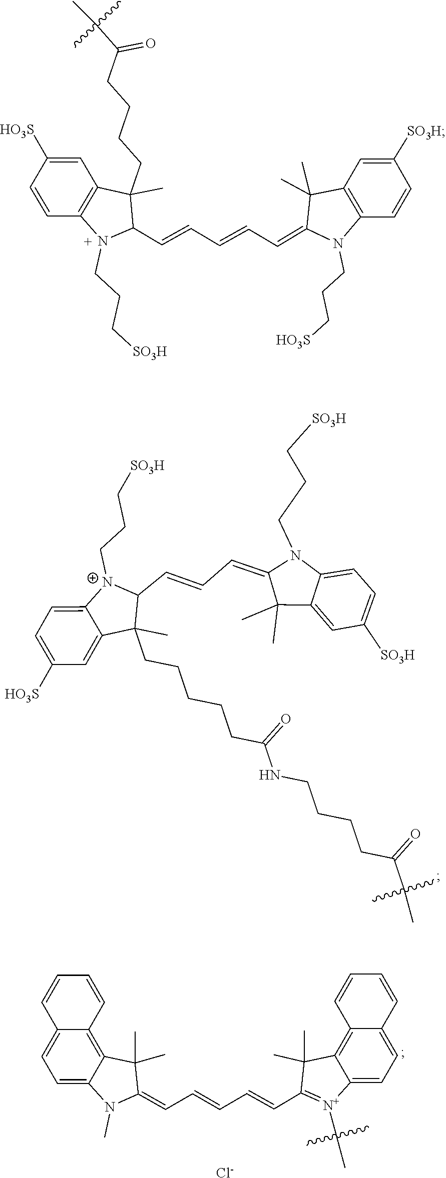 [SMILES: *C(=O)CCCCC1(C)c2cc(S(=O)(=O)O)ccc2[NH+](CCCS(=O)(=O)O)C1/C=C/C=C/C=C1/N(CCCS(=O)(=O)O)c2ccc(S(=O)(=O)O)cc2C1(C)C ; *C(=O)CCCCNC(=O)CCCCCC1(C)c2cc(S(=O)(=O)O)ccc2[NH+](CCCS(=O)(=O)O)C1/C=C/C=C1/N(CCCS(=O)(=O)O)c2ccc(S(=O)(=O)O)cc2C1(C)C ; *[N+]1=C(/C=C/C=C/C=C2/N(C)c3ccc4ccccc4c3C2(C)C)C(C)(C)c2c1ccc1ccccc21 ; [Cl-]]

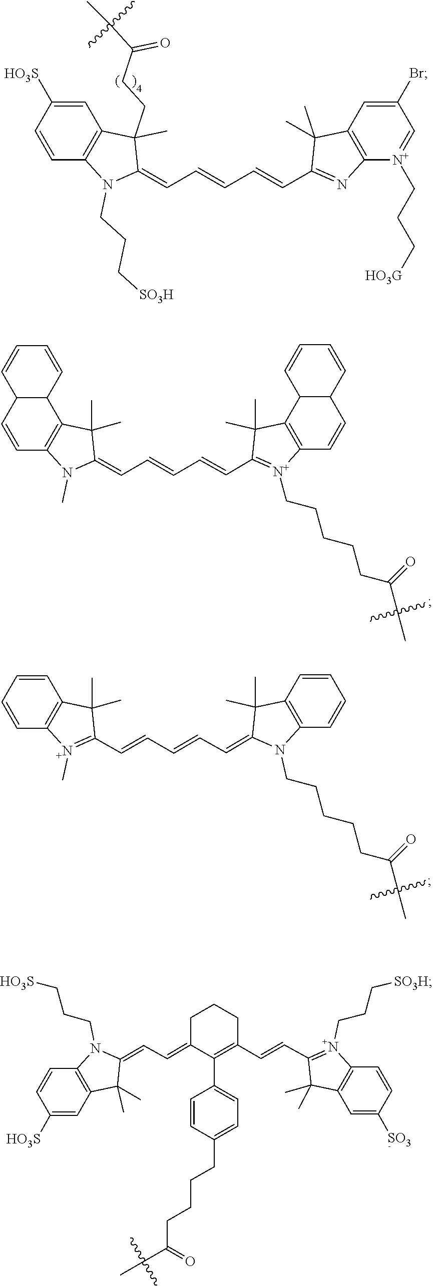

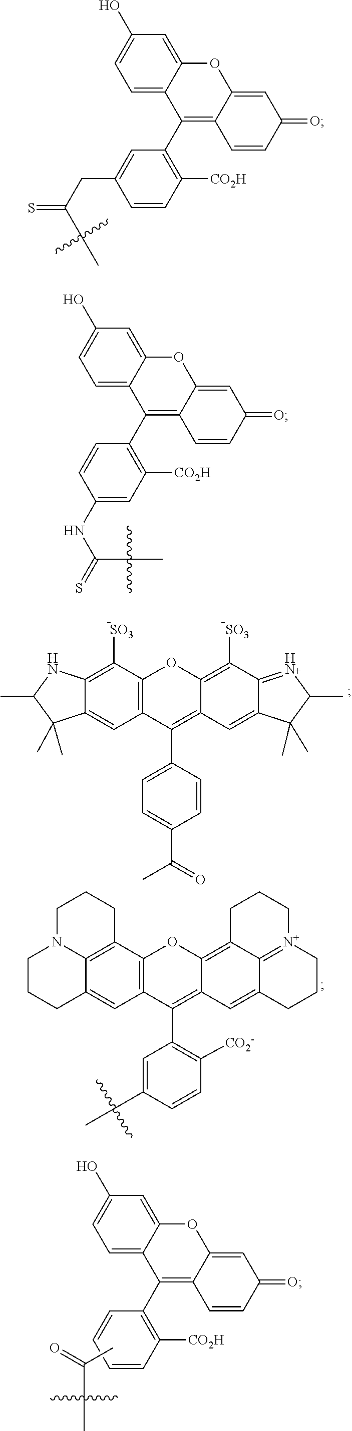

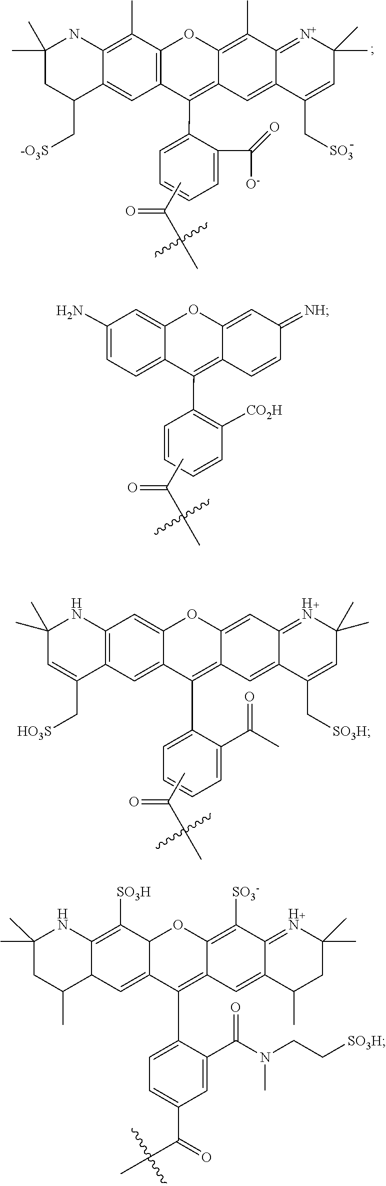

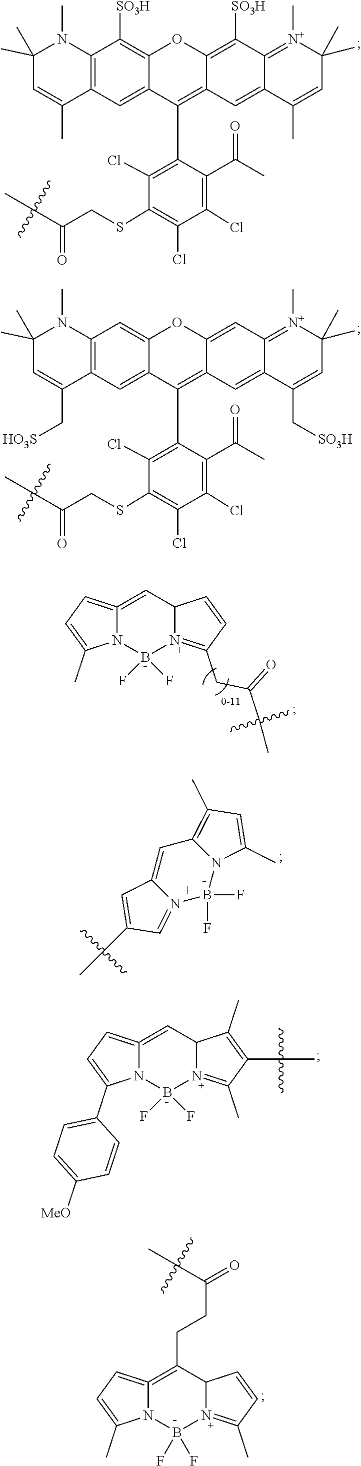

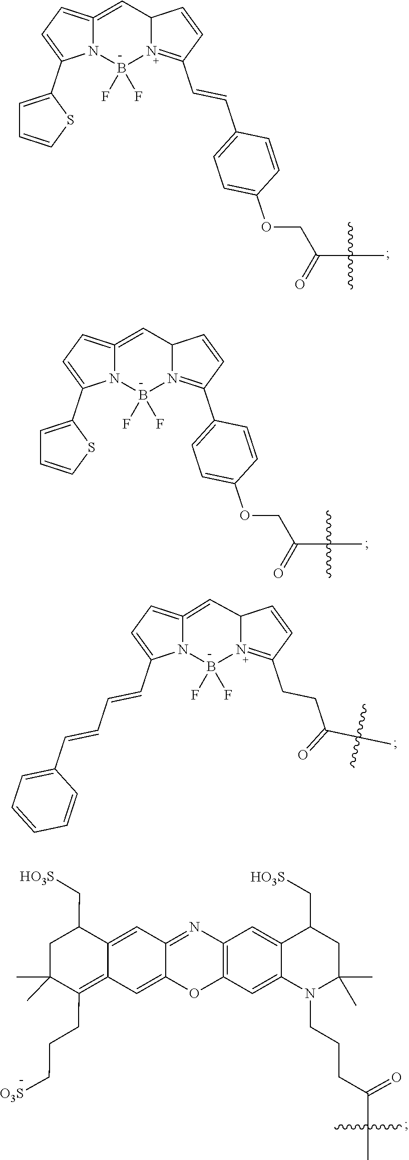

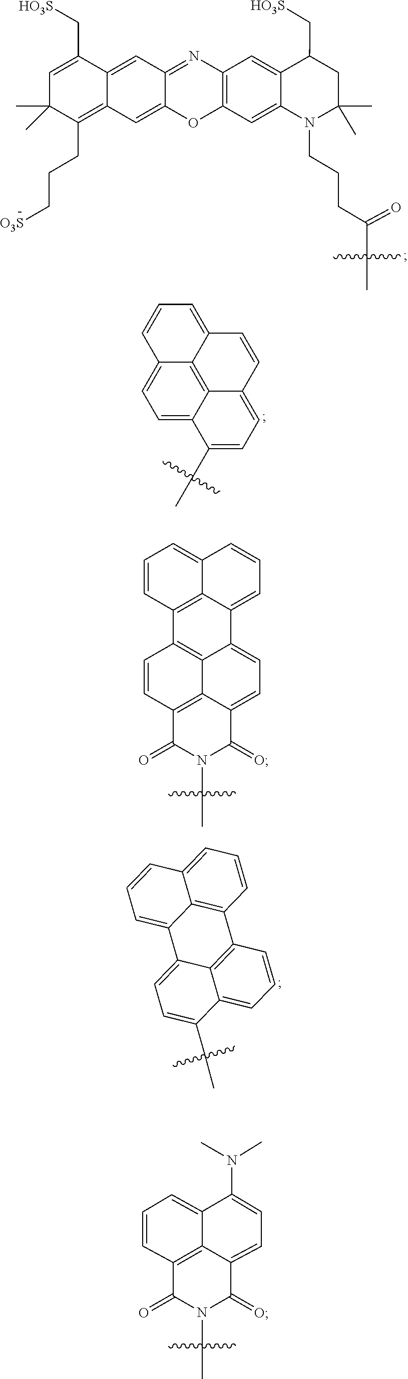

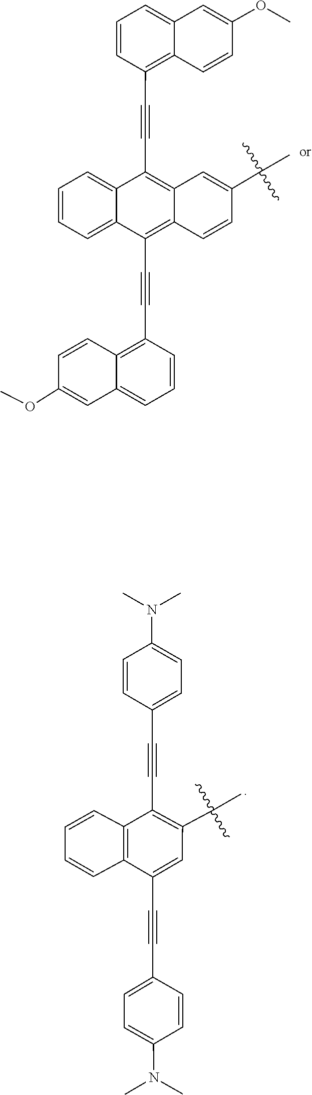

One embodiment provides a method of preparing a compound, the method comprising contacting a compound of Structure (II) with 1,4,-dithiothreitol, the compound of Structure (II) having the following structure:

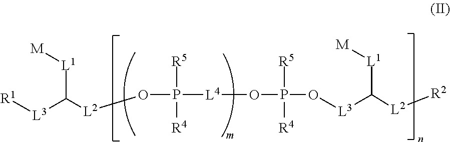

or a stereoisomer, salt or tautomer thereof, wherein:
M is, at each occurrence, independently a chromophore or fluorophore;
L¹ is at each occurrence, independently either: i) an optional alkylene, alkenylene, alkynylene, heteroalkylene, heteroalkenylene, heteroalkynylene or heteroatomic linker; or ii) a linker comprising a functional group capable of formation by reaction of two complementary reactive groups;
L² and L³ are, at each occurrence, independently an optional alkylene, heteroalkylene, or heteroatomic linker;
L⁴ is, at each occurrence, independently an alkylene or heteroalkylene linker;
R¹ and R² are each independently H, OH, SH, alkyl, alkoxy, alkylether, heteroalkyl, —OP(═Rₐ)(R_{b})R_{c}, Q, or a protected form thereof, or L′;
R⁴ is, at each occurrence, independently OH, SH, O⁻, S⁻, OR_{d} or SR_{d};
R⁵ is, at each occurrence, independently oxo, thioxo or absent;
Rₐ is O or S;
R_{b} is OH, SH, O⁻, S⁻, OR_{d} or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;
R_{d} is a counter ion;
Q is, at each occurrence, independently a moiety comprising a reactive group, or protected form thereof, capable of forming a covalent bond with an analyte molecule, a targeting moiety, a solid support or a complementary reactive group Q′;
L′ is, at each occurrence, independently a linker comprising a covalent bond to Q, a linker comprising a covalent bond to a targeting moiety, a linker comprising a covalent bond to an analyte molecule, a linker comprising a covalent bond to a solid support, a linker comprising a covalent bond to a solid support residue, a linker comprising a covalent bond to a nucleoside or a linker comprising a covalent bond to a further compound of Structure (II);
m is, at each occurrence, independently an integer of zero or greater, provided that at least one occurrence of m is an integer of one or greater; such that the compound includes at least one L⁴; and
n is an integer of one or greater,
   provided that,
at least one of R¹-L³- or R²-L²- comprises a —S—S—* moiety, wherein * represents a terminal end of the compound of Structure (II).

In some embodiments, L⁴ is, at each occurrence, independently a polyethylene oxide linker.

In some more specific embodiments of Structure (II), at least one occurrence of M is a fluorescent or colored moiety. In some embodiments, at least two occurrences of M are fluorescent or colored moieties.

In some embodiments, the contacting thereby converts the —S—S—* moiety to a —S—H moiety.

Still another embodiment provides a method of preparing a compound, the method comprising contacting a compound of Structure (III) with 1,4,-dithiothreitol, the compound of Structure (III) having the following structure:

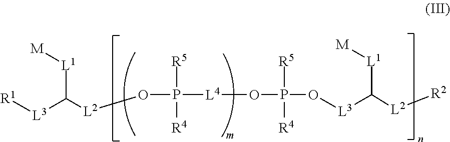

or a stereoisomer, salt, or tautomer thereof, wherein:
M is, at each occurrence, independently a chromophore, provided that at least one occurrence of M is a FRET donor, and another occurrence of M is a corresponding FRET acceptor;
L¹ is, at each occurrence, an optional linker;
L² and L³ are, at each occurrence, independently an optional alkylene, heteroalkylene, or heteroatomic linker;
L⁴ is, at each occurrence, independently an alkylene or heteroalkylene linker;
R¹ and R² are each independently H, OH, SH, alkyl, alkoxy, alkylether, heteroalkyl, —OP(═Rₐ)(R_{b})R_{c}, Q, or a protected form thereof, or L′;
R⁴ is, at each occurrence, independently OH, SH, O⁻, S⁻, OR_{d} or SR_{d};
R⁵ is, at each occurrence, independently oxo, thioxo or absent;
Rₐ is O or S;
R_{b} is OH, SH, O⁻, S⁻, OR_{d} or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;
R_{d} is a counter ion;
Q is, at each occurrence, independently a moiety comprising a reactive group, or protected form thereof, capable of forming a covalent bond with an analyte molecule, a targeting moiety, a solid support, or a complementary reactive group Q′;
L′ is, at each occurrence, independently a linker comprising a covalent bond to Q, a linker comprising a covalent bond to a targeting moiety, a linker comprising a covalent bond to an analyte molecule, a linker comprising a covalent bond to a solid support, a linker comprising a covalent bond to a solid support residue, a linker comprising a covalent bond to a nucleoside or a linker comprising a covalent bond to a further compound of Structure (III);
m is, at each occurrence, independently an integer of zero or greater; and
n is an integer of one or greater,
   provided that,
at least one of R¹-L³- or R²-L²- comprises a —S—S—* moiety, wherein * represents a terminal end of the compound of Structure (III).

In some embodiments, L⁴ is, at each occurrence, independently a polyethylene oxide linker.

In some embodiments of Structure (III), at least one occurrence of M is a fluorescent or colored moiety. In some embodiments, at least two occurrences of M are fluorescent or colored moieties.

In some embodiments, the contacting thereby converts the —S—S—* moiety to a —S—H moiety.

In some embodiments, Q has one of the exemplary Q moieties are provided in Table A below.

TABLE A
Exemplary Q Moieties

| Structure | Class |
|---|---|
| 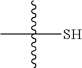 [SMILES: *S] | Sulfhydryl |
| 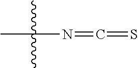 [SMILES: *N=C=S] | Isothiocyanate |
| 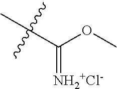 | Imidoester |
| 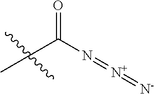 [SMILES: *C(=O)N=[N+]=[N-]] | Acyl Azide |
| 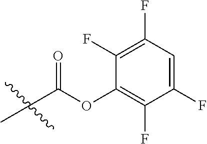 [SMILES: *C(=O)Oc1c(F)c(F)cc(F)c1F] | Activated Ester |
| 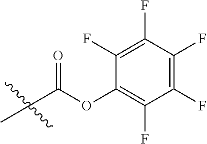 [SMILES: *C(=O)Oc1c(F)c(F)c(F)c(F)c1F] | Activated Ester |
| 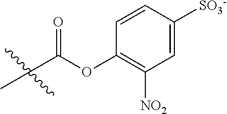 | Activated Ester |
| 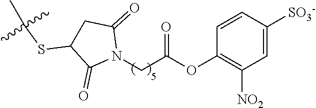 | Activated Ester |
| 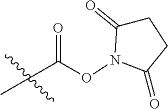 [SMILES: *C(=O)ON1C(=O)CCC1=O] | Activated Ester |
| 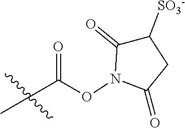 | Activated Ester |
| 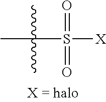 | Sulfonyl halide |
| 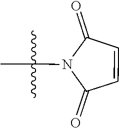 [SMILES: *N1C(=O)C=CC1=O] | Maleimide |
| 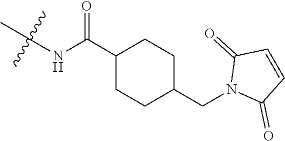 [SMILES: *NC(=O)C1CCC(CN2C(=O)C=CC2=O)CC1] | Maleimide |
| 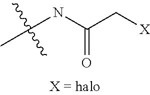 | α-haloimide |
| 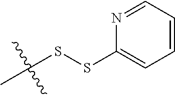 [SMILES: *SSc1ccccn1] | Disulfide |
| 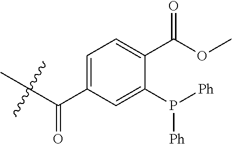 [SMILES: *C(=O)c1ccc(C(=O)OC)c(P(c2ccccc2)c2ccccc2)c1] | Phosphine |
| 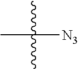 [SMILES: *N=[N+]=[N-]] | Azide |
| 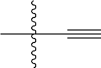 [SMILES: *C#C] | Alkyne |
| 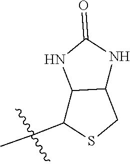 [SMILES: *C1SCC2NC(=O)NC21] | Biotin |
| 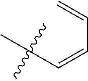 [SMILES: */C=C\C=C] | Diene |
| 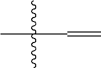 [SMILES: *C=C] | Alkene/ dienophile |
| 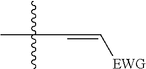 | Alkene/ dienophile |
| EWG = eletron withdrawing group | |
| —NH₂ | Amino |

In some embodiments, the solution further comprises magnesium chloride, dimethylsulfoxide, a polysorbate, or combinations thereof. In more specific embodiments, the polysorbate is polysorbate 20, polysorbate 40, polysorbate 60, polysorbate 80, or combinations thereof.

In more specific embodiments, the method further comprises heating the compound with 1,4,-dithiothreitol at an incubation temperature for an incubation time. In some embodiments, the incubation time is greater than 1 hour. In some embodiments, the incubation time is greater than 1.5 hours. In some embodiments, the incubation time is greater than 2 hours. In more specific embodiments, the incubation time is greater than about 0.5, 1.0, 1.25, 1.5, 1.75, 2.0, 2.25, 2.5, 2.75, 3.0, 3.25, 3.5, or 3.75 hours. In some embodiments, the incubation time is less than about 24, 12, 8, 6, 5, 4, or 3 hours.

In some embodiments, the incubation temperature is greater than 25° C. In more specific embodiments, the incubation temperature is greater than 30° C. In some embodiments, the incubation temperature is greater than 35° C. In some specific embodiments, the incubation temperature is about 37° C. and the incubation time is about 2 hours. In some embodiments, the incubation temperature is greater than about 15° C., 17° C., 20° C., 25° C., 27° C., 30° C., 32° C., 35° C., 37° C., or 40° C. In some embodiments, the incubation temperature is less than about 60° C., 55° C., 50° C., 45° C., 42° C., or 40° C.

In some embodiments, the method further comprises contacting the covalently bound cross-linking reagent with an analyte molecule thereby binding the compound to the analyte molecule in a sample mixture. In more specific embodiments, analyte molecule is selected from the group consisting of nucleic acids, carbohydrates, amino acids, polypeptides, glycoproteins, hormones, aptamers, and combinations thereof. In certain embodiments, the analyte molecule is selected from the group consisting of RNA, DNA, oligonucleotides, modified or derivatized nucleotides, enzymes, cell receptors, prions, cell receptor ligands, hormones, proteins, antibodies, antigens, toxins, bacteria, viruses, blood cells, tissue cells, and combinations thereof.

In more specific embodiments, the sample mixture comprises a sufficient amount of the compound bound to analyte molecules to produce an optical response when the sample mixture is illuminated at an excitation wavelength. In some embodiments, the excitation wavelength is between 450-750 nm, 450-500 nm, 500-550 nm, 550-600 nm, 600-650 nm, 650-700 nm, 700-750 nm, or 750-800 nm. In some more specific embodiments, the excitation wavelength is the excitation wavelength of one or more M moieties of the compound.

In certain embodiments, the optical response is a fluorescent response. In some embodiments, the sample comprises cells. In certain embodiments, the method further comprises observing the cells by flow cytometry. In some embodiments, the method further comprises distinguishing the fluorescence response from that of a second fluorophore having detectably different optical properties. In some embodiments, the method further comprises detecting the compound bound to the analyte molecule by its visible properties.

In some embodiments the polymer compound is not a peptide or protein. In some other embodiments, the polymer backbone has no amide bonds.

### Compound Preparation

The following Reaction Scheme illustrate exemplary methods of making compounds of this disclosure (compounds of Structure (I), (II), or (III)). It is understood that one skilled in the art may be able to make these compounds by similar methods or by combining other methods known to one skilled in the art. It is also understood that one skilled in the art would be able to make, in a similar manner as described below, other compounds of Structure (I), (II), or (III) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Advanced Organic Chemistry: Reactions, Mechanisms, and Structure, 5th edition (Wiley, December 2000)) or prepared as described in this disclosure.

DNA synthesis methodology can be applied to build compounds of Structure (I), (II), or (III). Monomers (e.g., phosphoramidite monomers) can be purchased commercially (e.g., from ChemGenes Corporation, Wilmington Mass.) or synthesized using methods known in the art (see, e.g., U.S. Publication No. 2019/0136065, which is hereby incorporated by reference). Introduction of desired moieties can be accomplished during the DNA synthesis steps by including the desired moiety as a portion of the monomer (see, e.g., L and M of the Representative DNA Synthesis Cycle, below). An exemplary DNA synthesis scheme is shown below.

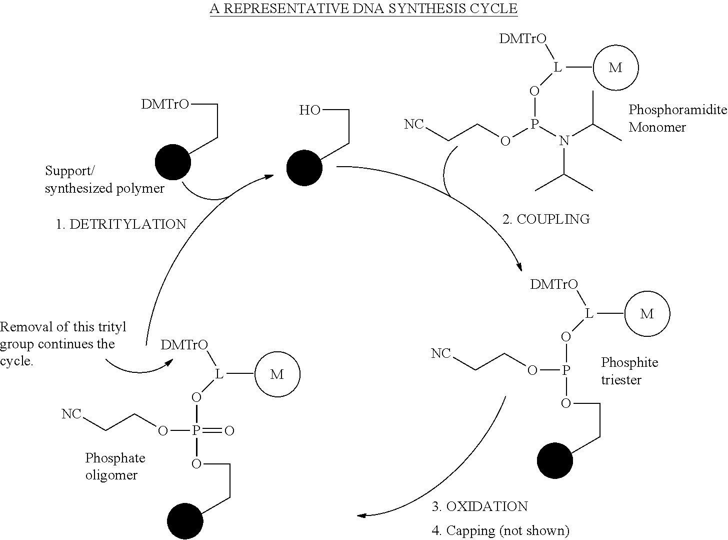

Oligomerization is initiated, typically, through the removal of a protecting group (e.g. a dimethoxytrityl group, DMTr) to reveal a free —OH (hydroxyl) group (Step 1, DETRITYLATION). In a subsequent coupling step, a phosphoramidite monomer is introduced that reacts with the free OH group making a new covalent bond to phosphorus, with concomitant loss of the diisopropyl amine group (Step 2, COUPLING). The resultant, phosphite triester is oxidized (e.g. with 12 and pyridine) to the more stable phosphate ester (Step 3, OXIDATION) and a capping step renders unreactive any remaining free OH groups (Step 4, CAPPING). The new product, phosphate oligomer, contains a DMTr protected OH group that can be deprotected to reinitiate the synthetic cycle so another phosphoramidite monomer can be appended to the oligomer.

Customization may occur at step 2 through the choice of phosphoramidite monomer. The nature of L (i.e., a linker group) and M (i.e., an optional fluorophore or chromophore moiety) in the scheme above are selected such that a desired compound of Structure (I), (II), or (III) is synthesized. M can be optionally absent to incorporate desired spacing between M moieties. A person of ordinary skill in the art can select multiple monomer types and combinations to arrive at compounds of the disclosure containing multiple fluorophores or chromophores with concurrent variability in linker groups.

Desired moieties and linker groups can be used to synthesize a desired compound of Structure (I), (II), or (III) by reaction under well-known (automated) DNA synthesis conditions. Generally, compounds having the following structure may be used:

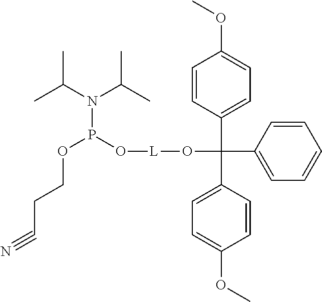

wherein:
L is a desired linker moiety (e.g., including PEG or a pendant dye-containing moiety).

In some specific embodiments, the following compound may be used in the synthesis of a compound of Structure (I), (II), or (III):

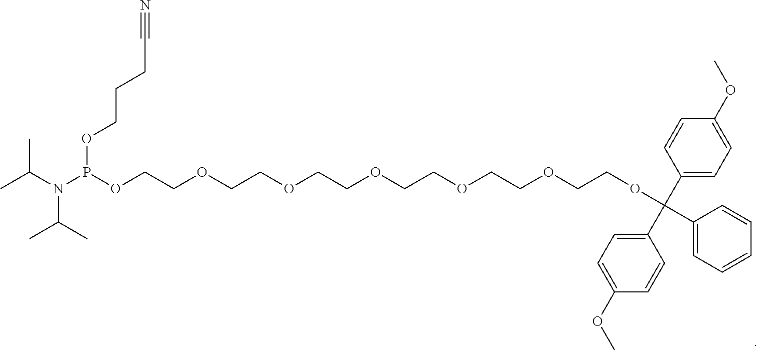 [SMILES: COc1ccc(C(OCCOCCOCCOCCOCCOCCOP(OCCCC#N)N(C(C)C)C(C)C)(c2ccccc2)c2ccc(OC)cc2)cc1]

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin, or a 2-chlorotrityl-chloride resin.

Furthermore, all compounds of the disclosure which exist in free base or acid form can be converted to their salts by treatment with the appropriate inorganic or organic base or acid by methods known to one skilled in the art. Salts of the compounds of the disclosure can be converted to their free base or acid form by standard techniques.

### Example 1

### Test Reduction Using TCEP Vs. DTT

A representative compound was tested to determine reduction conditions using tris(2-carboxyethyl)phosphine (TCEP) compared to 1,4-dithiothreitol (DTT). The compound was reduced according to the following reaction scheme:

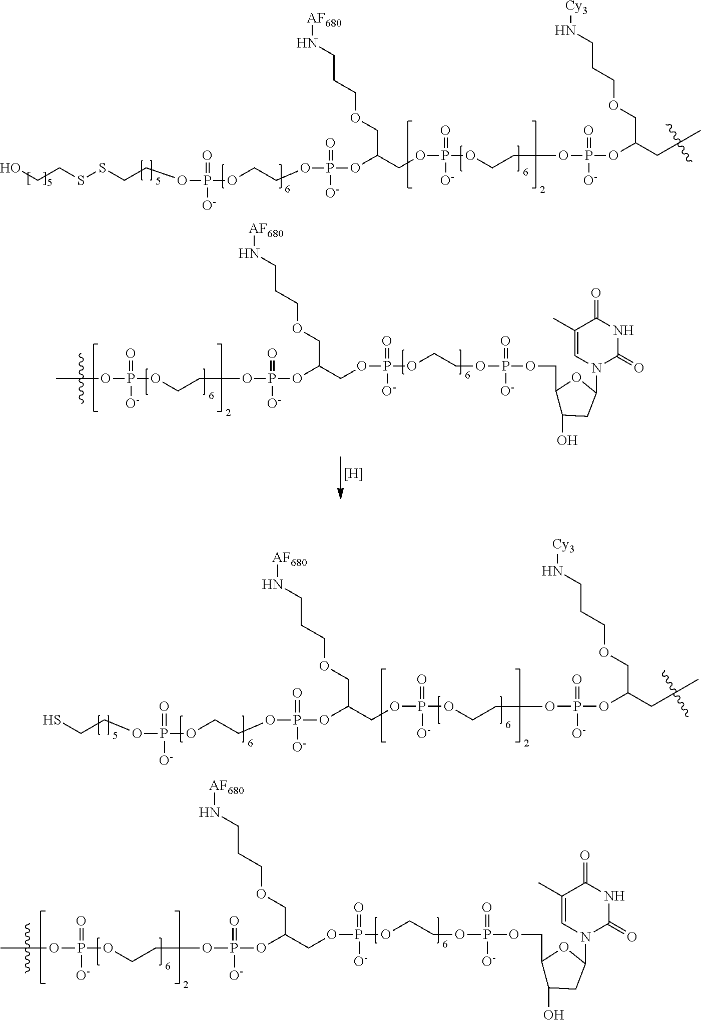

Cy₃ in the above scheme refers to a moiety has the following structure:

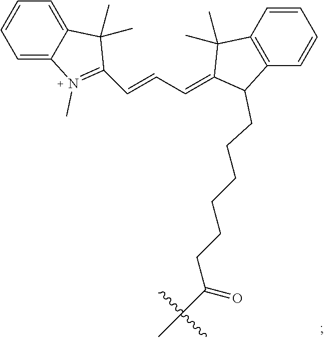

and

AF₆₈₀ in the above scheme refers to a moiety having the following structure:

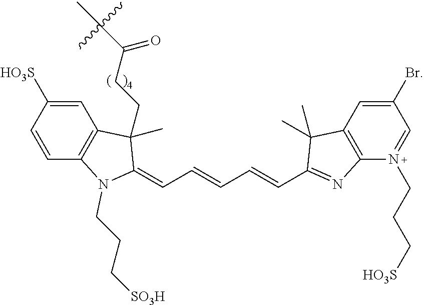

When the reduction was performed with TCEP, the mass spectrometry spectra showed a multitude of peaks. However, when the reduction was performed with DTT, the peaks were resolved into a single peak corresponding to the expected product. Following the reduction step, bis-maleimidoethane (or 1,1′-(ethane-1,2-diyl)bis(1H-pyrrole-2,5-dione)) was added under coupling conditions.

For the TCEP reduction, adding more TCEP (up to 10 molar equivalents) and polysorbate 20 helped the reduction reaction proceed, but complete reduction of the disulfide is not achieved; however addition of BMOE resulted in dimerization of the compound and a +18 additive (i.e., water).

For the DTT reduction, the disulfide is reduced completely and the dimer and the +18 additive are not observed.

Many additives and reaction conditions were also tested. These studies showed that, when using DTT, a pH of 7-7.5 was essential and 2-4 mM EDTA helped the reduction. When certain dyes are present, methanol and acetonitrile are helpful; additives such as MgCl₂ is helpful to prevent aggregation/dimerization in the final maleimide product with certain dyes present.

Reducing with DTT (20 molar equivalents) led to a more efficient activation once a cross-linker is added. There was no reformation of dimer or +18 additive when cross-linkers BMOE, BMH, BMB, and BM(PEG)₂ were used.

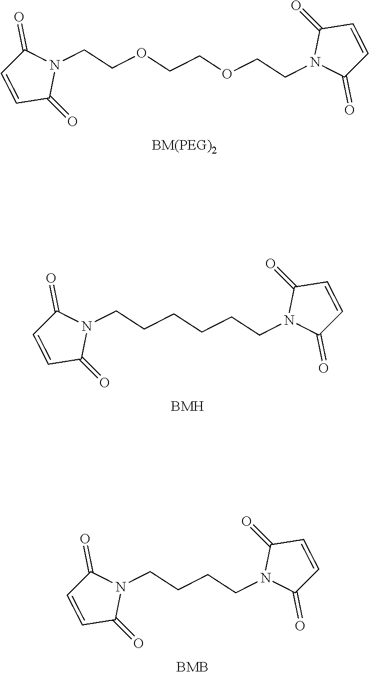 [SMILES: O=C1C=CC(=O)N1CCOCCOCCN1C(=O)C=CC1=O ; O=C1C=CC(=O)N1CCCCCCN1C(=O)C=CC1=O ; O=C1C=CC(=O)N1CCCCN1C(=O)C=CC1=O]

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Application No. 63/211,435, filed Jun. 16, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of preparing a compound, the method comprising contacting a compound of Structure (I) with 1,4,-dithiothreitol, the compound of Structure (I) having the following structure: 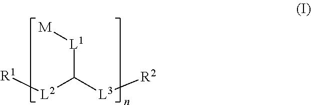
wherein:
M is, at each occurrence, independently a moiety comprising two or more double bonds and at least one degree of conjugation, provided that at least one occurrence of M is a cyanine or derivative thereof;
L¹, L², and L³ are, at each occurrence, independently optional linkers;
R¹ and R² are independently H, OH, SH, NH₂, alkyl, alkoxy, alkylether, hydroxylalkyl, aminoalkyl, hydroxylalkylether, sulfhydrylalkyl, —S—S-alkyl, —S—S-hydroxylalkyl, alkyl-S—S-alkyl, alkyl-S—S-hydroxylalkyl, phosphoalkyl-S—S-alkyl, phosphoalkyl-S—S-hydroxylalkyl, sulfyhdrylalkylether, phosphate, thiophosphate, alkylphospho, alkylthiophospho, —Oalkylphospho, —Oalkylthiophospho, alkyletherphospho, alkyletherthiophospho, —Oalkyletherphospho, —Oalkyletherthiophospho, phosphoalkyl, phosphoalkylether, thiophosphoalkyl, thiophosphoalkylether, —Ophosphoalkyl, —Ophosphoalkylether, —Othiophosphoalkyl, —Othiophosphoalkylether, or have the following structure: 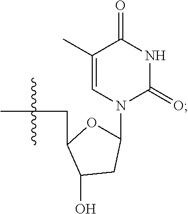 [SMILES: *CC1OC(n2cc(C)c(=O)[nH]c2=O)CC1O]
and
n is an integer greater than 0,
provided that,
at least one of R¹-L²- or R²-L³- comprises a —S—S—* moiety, wherein * represents a terminal end of the compound of Structure (I).

2. The method of claim 1, wherein the contacting thereby converts the —S—S—* moiety to a —S—H moiety.

3. The method of claim 1 or 2, wherein the contacting is performed in a solution having a pH between 6.5 and 8.

4. The method of any one of claims 1-3, wherein the contacting is performed in a solution having a pH between 7-7.5.

5. The method of any one of claims 3-4, wherein the solution comprises ethylenediaminetetraacetic acid (EDTA).

6. The method of any one of claims 3-5, wherein the solution comprises a concentration of ethylenediaminetetraacetic acid (EDTA) from about 1 to 6 mM.

7. The method of any one of claims 3-6, wherein the solution comprises a concentration of ethylenediaminetetraacetic acid (EDTA) from about 1.5 to 5.5 mM.

8. The method of any one of claims 3-7, wherein the solution comprises a concentration of ethylenediaminetetraacetic acid (EDTA) from about 1.9 to 4.2 mM.

9. The method of any one of claims 3-8, wherein the solution comprises a concentration of ethylenediaminetetraacetic acid (EDTA) from about 2 to 4 mM.

10. The method of any one of claims 3-9, wherein the solution comprises magnesium chloride (MgCl₂).

11. The method of any one of claims 3-10, wherein the solution comprises magnesium chloride (MgCl₂) at a concentration between about 1 to 50 mM.

12. The method of any one of claims 3-11, wherein the solution comprises magnesium chloride (MgCl₂) at a concentration between about 2 to 40 mM.

13. The method of any one of claims 3-12, wherein the solution comprises magnesium chloride (MgCl₂) at a concentration between about 3 to 35 mM.

14. The method of any one of claims 3-13, wherein the solution comprises magnesium chloride (MgCl₂) at a concentration between about 4 to 30 mM.

15. The method of any one of claims 3-14, wherein the solution comprises magnesium chloride (MgCl₂) at a concentration between about 5 to 25 mM.

16. The method of any one of claims 3-15, wherein the solution comprises an organic solvent.

17. The method of claim 16, wherein the organic solvent is methanol.

18. The method of claim 16, wherein the organic solvent is acetonitrile.

19. The method of any one of claims 16-18, wherein the solution has an organic solvent concentration greater than about 5% by volume.

20. The method of any one of claims 16-19, wherein the solution has an organic solvent concentration greater than about 7.5% by volume.

21. The method of any one of claims 16-20, wherein the solution has an organic solvent concentration greater than about 10% by volume.

22. The method of any one of claims 16-21, wherein the solution has an organic solvent concentration greater than about 15% by volume.

23. The method of any one of claims 2-22, wherein the method further comprises contacting the —S—H moiety with a cross-linking reagent, thereby forming a covalent bond between the compound and the cross-linking reagent.

24. The method of claim 23, wherein the cross-linking reagent has the following structure: 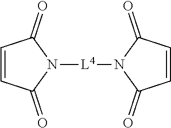
wherein:
L⁴ is a linker.

25. The method of claim 24, wherein L⁴ comprises atoms selected from C and O.

26. The method of claim 24 or 25, wherein L⁴ has one of the following structures: 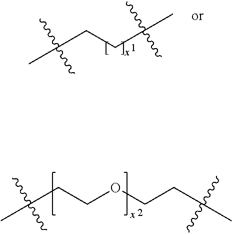
wherein:
x¹ and x² are an integer greater than 0.

27. The method of claim 26, wherein x¹ is 1.

28. The method of claim 26, wherein x¹ is 3.

29. The method of claim 26, wherein x¹ is 5.

30. The method of claim 26, wherein x² is 2.

31. The method of claim 26, wherein x² is 3.

32. The method of claim 23, wherein the cross-linking reagent has one of the following structures:

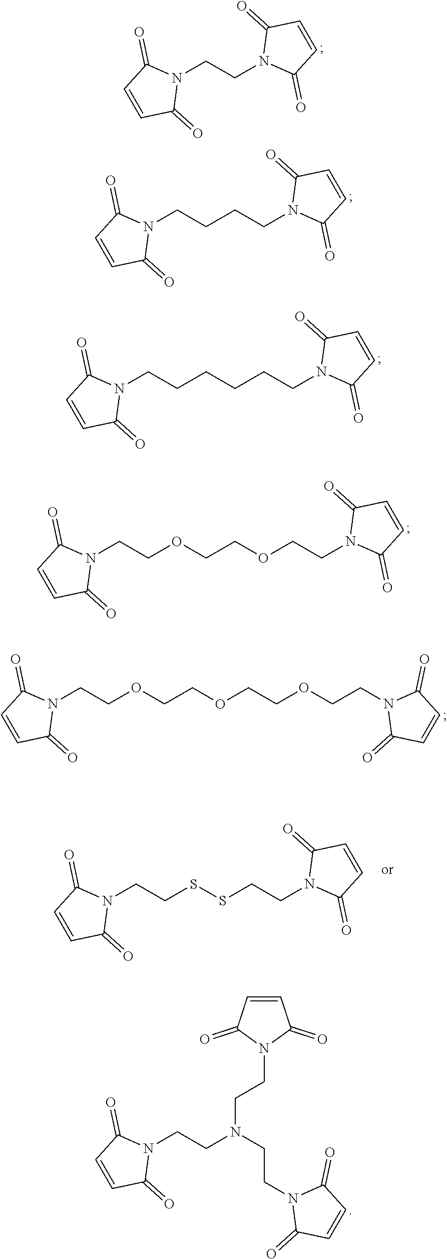

33. The method of any one of claims 1-32, wherein each occurrence of L¹ comprises atoms selected from C, O, S, N and P.

34. The method of any one of claims 1-33, wherein each occurrence of L² comprises atoms selected from C, O, S, N and P.

35. The method of any one of claims 1-34, wherein each occurrence of L³ comprises atoms selected from C, O, S, N and P.

36. The method of any one of claims 1-35, wherein at least one occurrence of L¹ comprises atoms selected from C, O, and N.

37. The method of any one of claims 1-36, wherein at least one occurrence of L¹ is an alkylene or heteroalkylene linker.

38. The method of any one of claims 1-37, wherein at least one occurrence of L¹ is an alkylene linker.

39. The method of any one of claims 1-38, wherein at least one occurrence of L¹ is a heteroalkylene linker.

40. The method of any one of claims 1-39, wherein at least one occurrence of L² has the following structure: 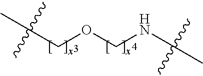
wherein:
x³ and x⁴ are each independently a integer greater than 0.

41. The method of claim 40, wherein x³ is 1, 2, 3, or 4.

42. The method of claim 40 or 41, wherein x⁴ is 2, 3, 4, or 5.

43. The method of any one of claims 40-42, wherein x³ is 1 or 2 and x⁴ is 2, 3, or 4.

44. The method of any one of claims 1-43, wherein at least one occurrence of L¹ has one of the following structures:

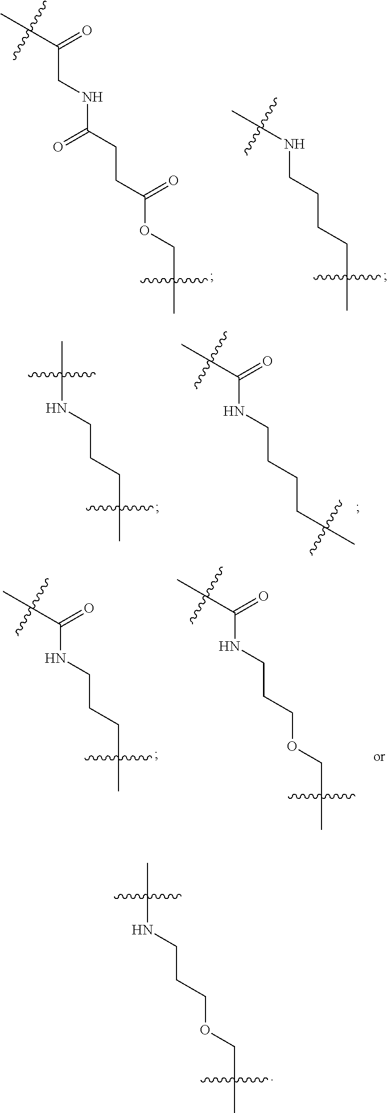

45. The method of any one of claims 1-37, wherein each occurrence of L¹ has one of the following structures:

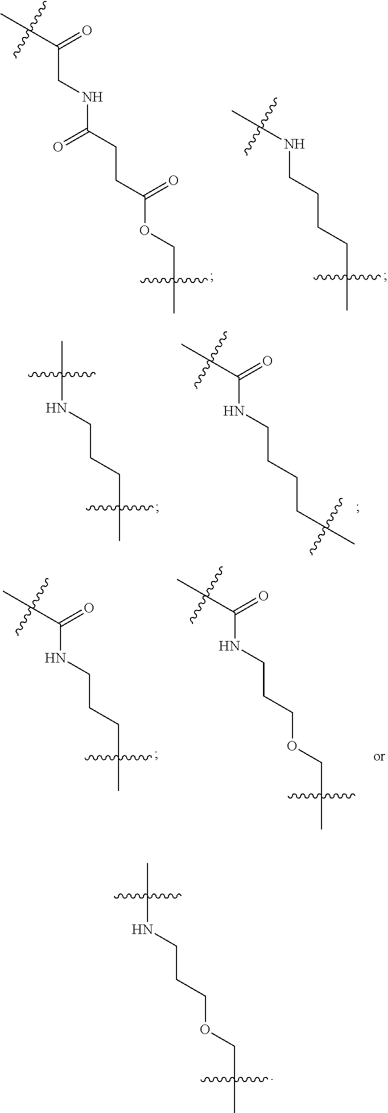

46. The method of any one of claims 1-45, wherein n is an integer from 1-20.

47. The method of any one of claims 1-46, wherein n is an integer from 2-10.

48. The method of any one of claims 1-47, wherein at least one occurrence of L² comprises atoms selected from C, O, S, and P.

49. The method of any one of claims 1-48, wherein at least one occurrence of L² is alkylene or heteroalkylene.

50. The method of any one of claims 1-49, wherein at least one occurrence of L² comprises the following structure:

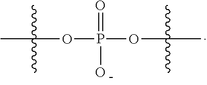 [SMILES: *OP(=O)(O)O*]

51. The method of any one of claims 1-50, wherein at least one occurrence of L² comprises the following structure: 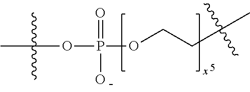
wherein:
x⁵ is an integer greater than 0.

52. The method of claim 51, wherein x⁵ is 2, 3, or 6.

53. The method of any one of claims 1-52, wherein at least one occurrence of L² comprises the following structure:

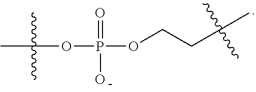 [SMILES: *CCOP(=O)(O)O*]

54. The method of any one of claims 1-53, wherein at least one occurrence of L³ comprises atoms selected from C, O, S, and P.

55. The method of any one of claims 1-54, wherein at least one occurrence of L³ is alkylene or heteroalkylene.

56. The method of any one of claims 1-55, wherein at least one occurrence of L³ comprises the following structure:

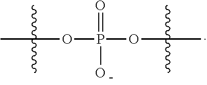 [SMILES: *OP(=O)(O)O*]

57. The method of any one of claims 1-56, wherein at least one occurrence of L³ comprises the following structure: 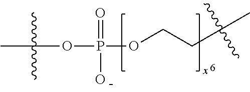
wherein:
x⁶ is an integer greater than 0.

58. The method of claim 57, wherein x⁶ is 2, 3, or 6.

59. The method of any one of claims 1-58, wherein at least one occurrence of L³ comprises the following structure:

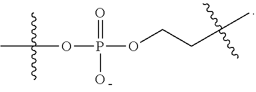 [SMILES: *CCOP(=O)(O)O*]

60. The method of any one of claims 1-58, wherein at least one occurrence of L³ comprises the following structure: 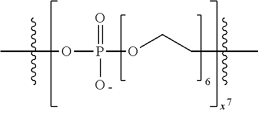
wherein:
x⁷ is an integer greater than 0.

61. The method of claim 60, wherein x⁷ is 1, 2, 3, 4, 5, or 6.

62. The method of claim 60 or 61, wherein x⁷ is 3.

63. The method of any one of claims 1-62, wherein R¹ is H, OH, SH, NH₂, alkyl, alkylether, hydroxylalkyl, aminoalkyl, hydroxylalkylether, sulfhydrylalkyl, —S—S-alkyl, —S—S-hydroxylalkyl, alkyl-S—S-alkyl, alkyl-S—S-hydroxylalkyl, phosphoalkyl-S—S-alkyl, phosphoalkyl-S—S-hydroxylalkyl, sulfyhdrylalkylether, phosphate, thiophosphate, or alkylphospho.

64. The method of any one of claims 1-63, wherein R² is H, OH, SH, NH₂, alkyl, alkylether, hydroxylalkyl, aminoalkyl, hydroxylalkylether, sulfhydrylalkyl, —S—S-alkyl, —S—S-hydroxylalkyl, alkyl-S—S-alkyl, alkyl-S—S-hydroxylalkyl, phosphoalkyl-S—S-alkyl, phosphoalkyl-S—S-hydroxylalkyl, sulfyhdrylalkylether, phosphate, thiophosphate, alkylphospho, or has the following structure:

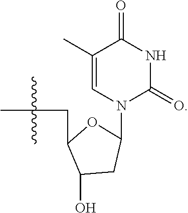 [SMILES: *CC1OC(n2cc(C)c(=O)[nH]c2=O)CC1O]

65. The method of any one of claims 1-64, wherein R¹ comprises the following structure: 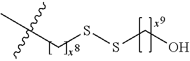
wherein:
x⁸ and x⁹ are each independently an integer greater than 0.

66. The method of claim 65, wherein x⁸ is 1, 2, 3, 4, 5, 6, 7, 8, 9, 10, 11, or 12.

67. The method of claim 65 or 66, wherein x⁹ is 1, 2, 3, 4, 5, 6, 7, 8, 9, 10, 11, or 12.

68. The method of any one of claims 65-67, wherein x⁸ is 4, 5, 6, 7, or 8 and x⁹ is 4, 5, 6, 7, or 8.

69. The method of any one of claims 1-68, wherein at least one occurrence of M is a fluorophore or chromophore.

70. The method of any one of claims 1-69, wherein two or more occurrences of M form a FRET donor-acceptor pair.

71. The method of any one of claims 1-70, wherein at least one occurrence of M is a moiety comprising three or more aryl or heteroaryl rings, or combinations thereof.

72. The method of any one of claims 1-71, wherein M is, at each occurrence, independently selected from the group consisting of coumarin dye, resorufin dye, dipyrrometheneboron difluoride dye, ruthenium bipyridyl dye, thiazole orange dye, polymethine, N-aryl-1,8-naphthalimide dye, boron-dipyrromethene, rhodamine, a cyanine dye, pyrene, perylene, perylene monoimide, 6-FAM, 5-FAM, 6-FITC, 5-FITC, and derivatives thereof.

73. The method of any one of claims 1-72, wherein at least one occurrence of M has one of the following structures:

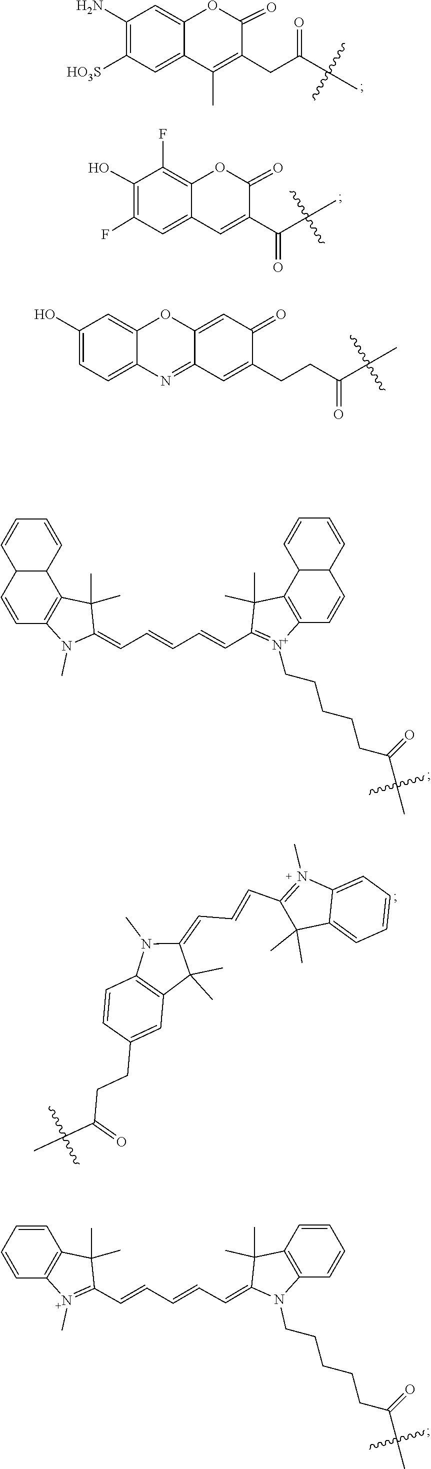

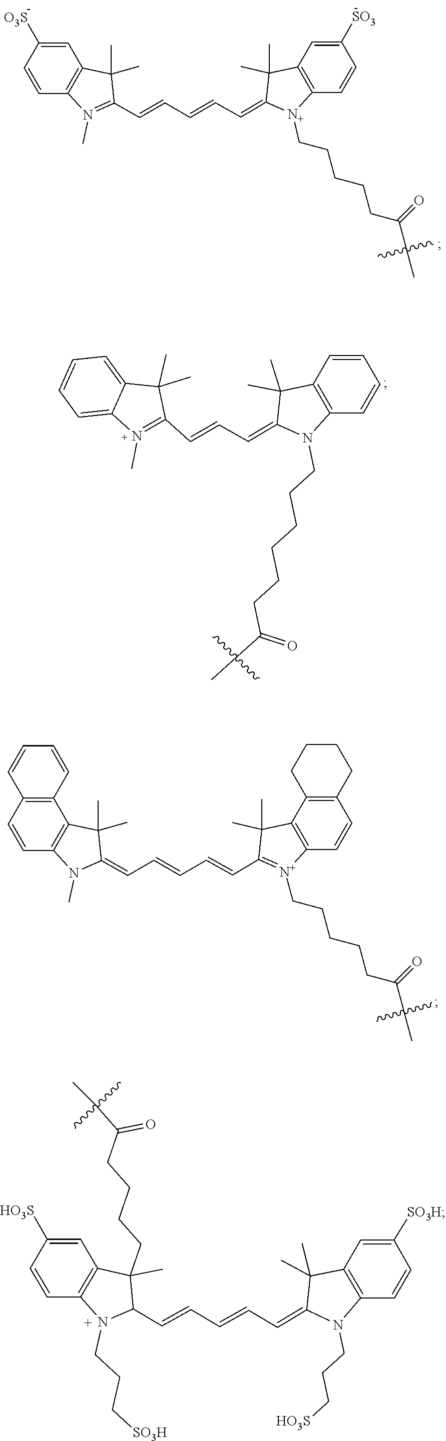

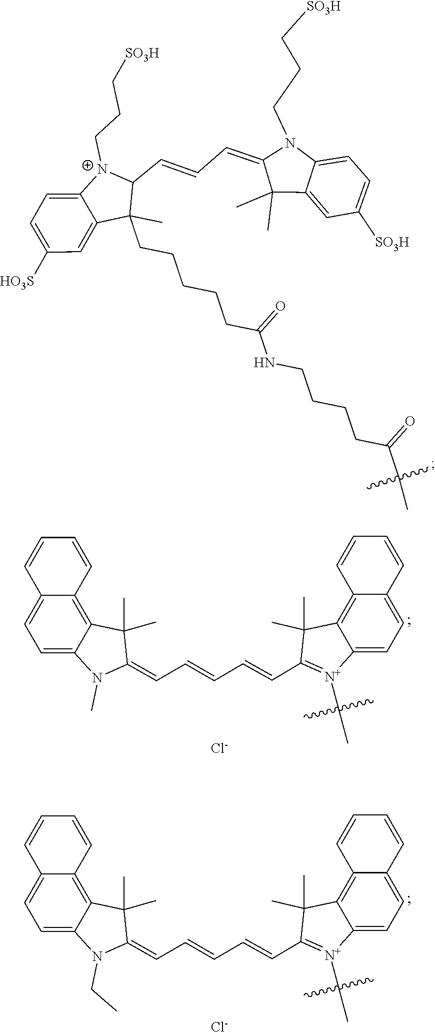 [SMILES: *C(=O)CCCCNC(=O)CCCCCC1(C)c2cc(S(=O)(=O)O)ccc2[NH+](CCCS(=O)(=O)O)C1/C=C/C=C1/N(CCCS(=O)(=O)O)c2ccc(S(=O)(=O)O)cc2C1(C)C ; *[N+]1=C(/C=C/C=C/C=C2/N(C)c3ccc4ccccc4c3C2(C)C)C(C)(C)c2c1ccc1ccccc21 ; [Cl-] ; *[N+]1=C(/C=C/C=C/C=C2/N(CC)c3ccc4ccccc4c3C2(C)C)C(C)(C)c2c1ccc1ccccc21 ; [Cl-]]

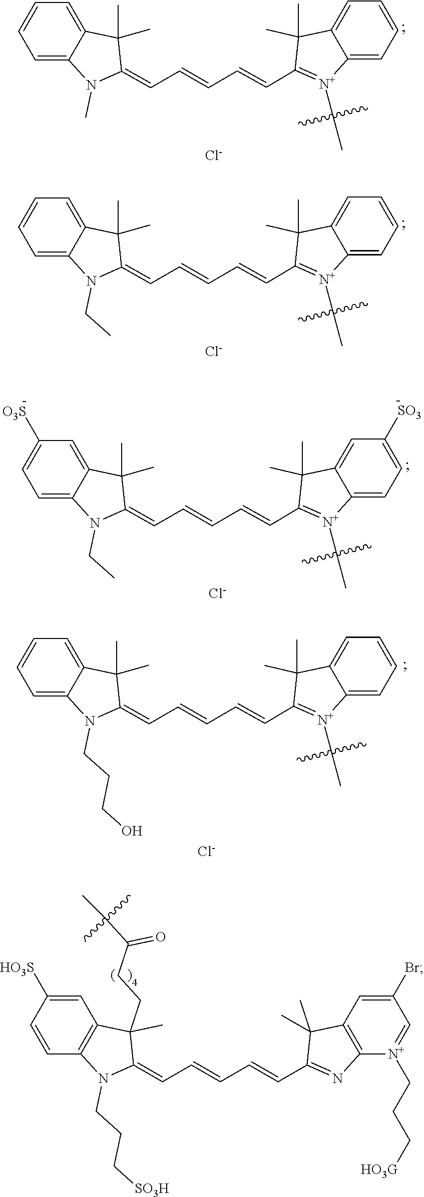

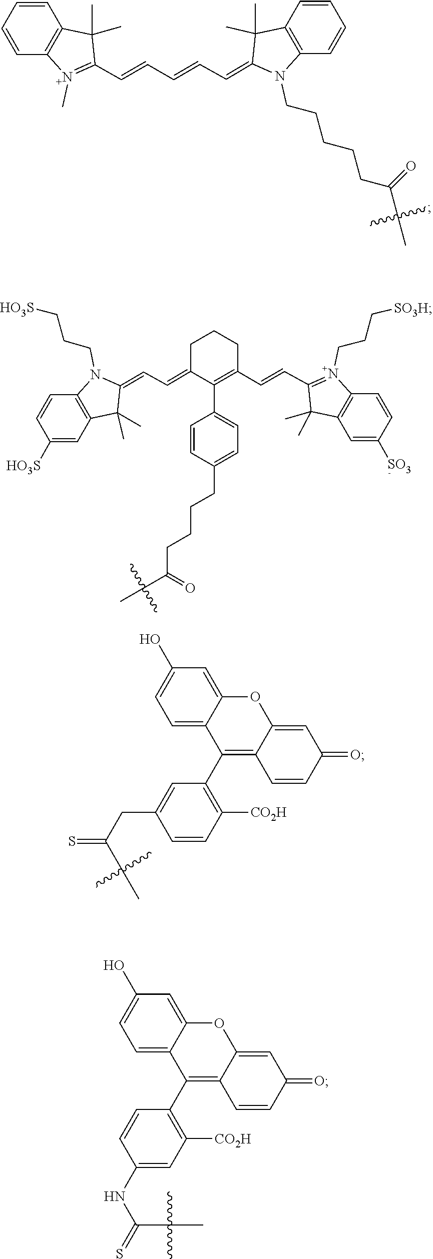

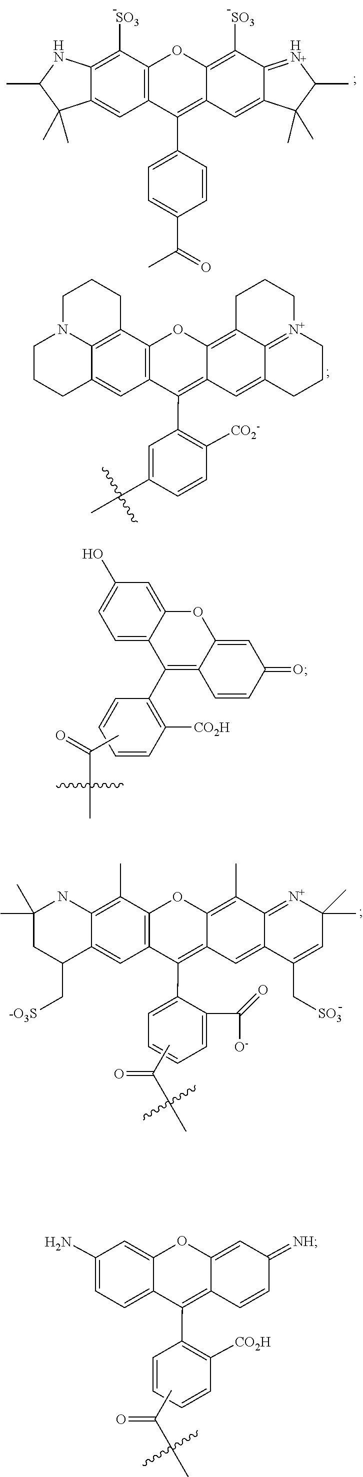

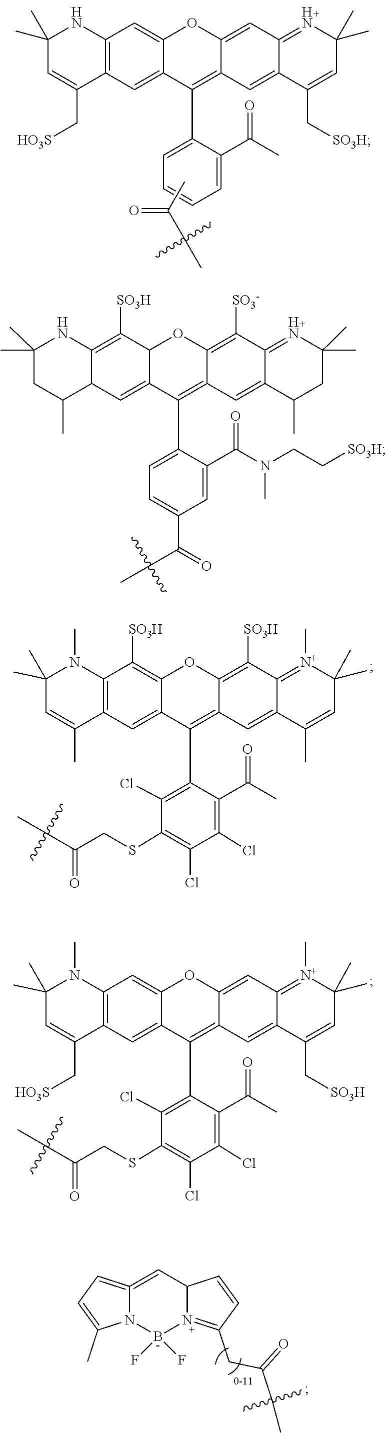

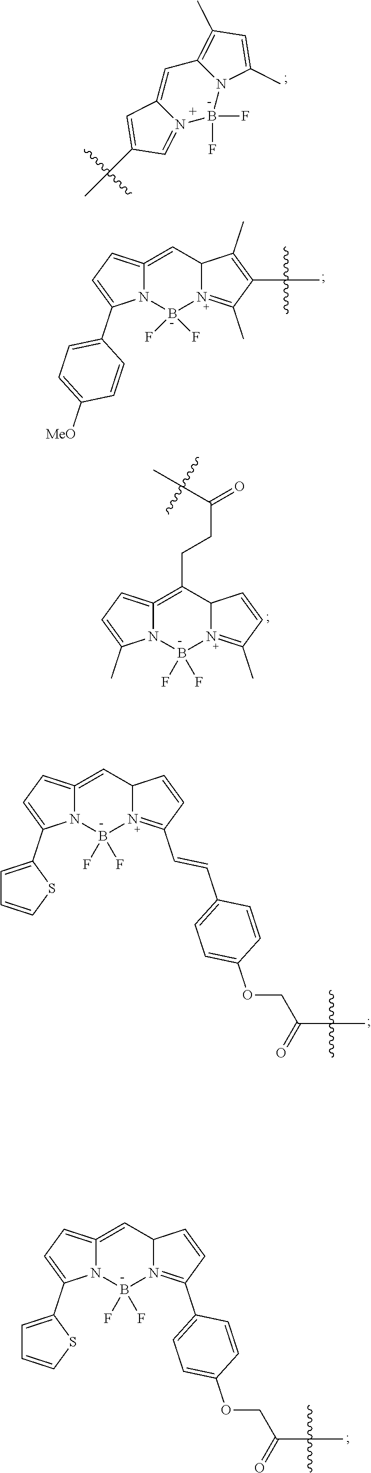

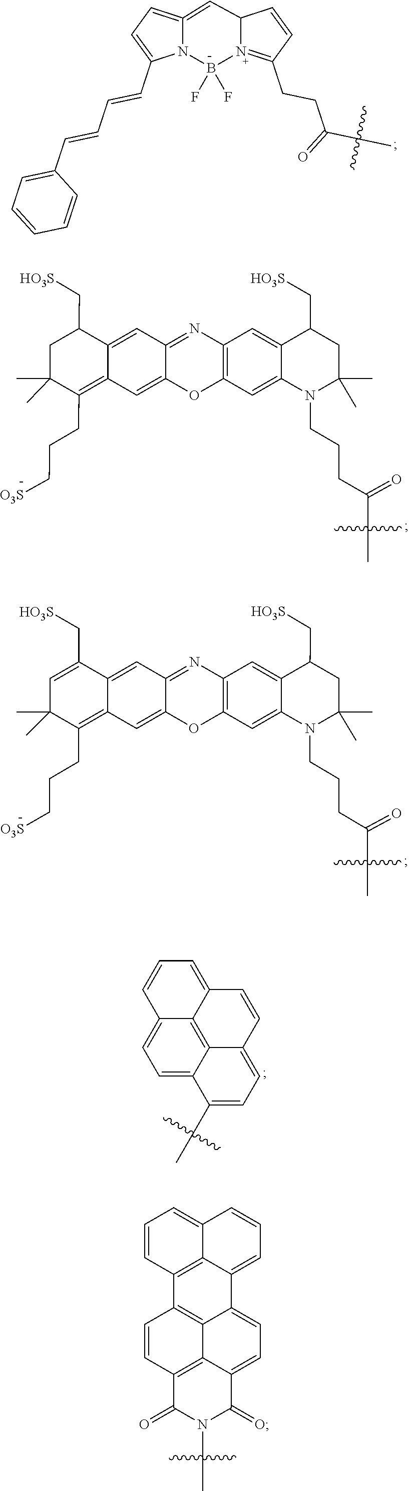

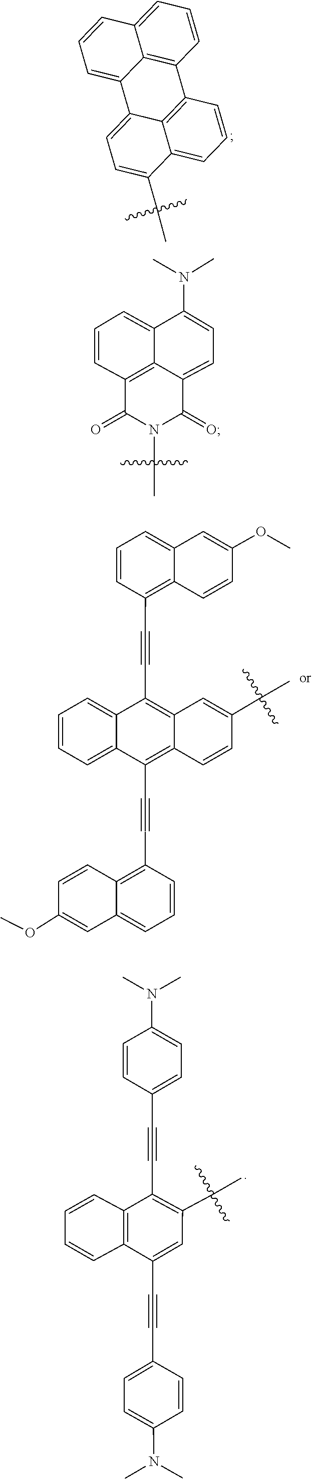

74. The method of any one of claims 3-73, wherein the solution further comprises magnesium chloride, dimethylsulfoxide, a polysorbate, or combinations thereof.

75. The method of any one of claims 1-74, wherein the method further comprises heating the compound with 1,4,-dithiothreitol at an incubation temperature for an incubation time.

76. The method of claim 75, wherein the incubation time is greater than 1 hour.

77. The method of claim 75 or 76, wherein the incubation time is greater than 1.5 hours.

78. The method of any one of claims 75-77, wherein the incubation time is greater than 2 hours.

79. The method of any one of claims 75-78, wherein the incubation temperature is greater than 25° C.

80. The method of any one of claims 75-79, wherein the incubation temperature is greater than 30° C.

81. The method of any one of claims 75-80, wherein the incubation temperature is greater than 35° C.

82. The method of any one of claims 75-81, wherein the incubation temperature is about 37° C. and the incubation time is about 2 hours.

83. The method of any one of claims 7-82, wherein the method further comprises contacting the covalently bound cross-linking reagent with an analyte molecule thereby binding the compound to the analyte molecule in a sample mixture.

84. The method of claim 83, wherein the analyte molecule is selected from the group consisting of nucleic acids, carbohydrates, amino acids, polypeptides, glycoproteins, hormones, aptamers, and combinations thereof.

85. The method of claim 83, wherein the analyte molecule is selected from the group consisting of RNA, DNA, oligonucleotides, modified or derivatized nucleotides, enzymes, cell receptors, prions, cell receptor ligands, hormones, proteins, antibodies, antigens, toxins, bacteria, viruses, blood cells, tissue cells, and combinations thereof.

86. The method of any one of claims 83-85, wherein the sample mixture comprises a sufficient amount of the compound bound to analyte molecules to produce an optical response when the sample mixture is illuminated at an excitation wavelength.

87. The method of claim 86, wherein the excitation wavelength is the excitation wavelength of one or more M moieties of the compound.

88. The method of claim 86 or 87, wherein the optical response is a fluorescent response.

89. The method of claims 86-88, wherein the sample comprises cells.

90. The method of claim 89, wherein the method further comprises observing the cells by flow cytometry.

91. The method of claim 88, wherein the method further comprises distinguishing the fluorescence response from that of a second fluorophore having detectably different optical properties.

92. The method of any one of claims 83-85, wherein the method further comprises detecting the compound bound to the analyte molecule by its visible properties.
